(19) United States
(12) Patent Application Publication
(10) Pub. No.: US 2023/0181604 A1
(43) Pub. Date: Jun. 15, 2023

(54) Use of Mitochondria-Targeted Antioxidants to Treat Severe Inflammatory Conditions

(71) Applicant: Mitotech S.A., Luxembourg (LU)

(72) Inventors: Maxim V. SKULACHEV, Moscow (RU); Roman ZINOVKIN, Moscow (RU); Alexander ANDREEV-ANDRIEVSKY, Moscow (RU); Maxim EGOROV, Moscow (RU); Elena KARGER, Moscow (RU); Anton PETROV, Luxembourg (LU); Lawrence FRIEDHOFF, River Vale, NJ (US)

(21) Appl. No.: 17/916,702

(22) PCT Filed: Apr. 5, 2021
(86) PCT No.: PCT/IB2021/000219

§ 371 Date: Oct. 3, 2022

Related U.S. Application Data
(60) Provisional application No. 63/004,893, filed on Apr. 3, 2020.
(60) Provisional application No. 63/011,920, filed on Apr. 17, 2020.

(51) Int. Cl. A61K 31/66; A61K 31/353; A61K 31/335; A61K 31/4375; A61P 31/16; A61K 45/06

(52) U.S. Cl. CPC A61K 31/66; A61K 31/353; A61K 31/335; A61K 31/4375; A61P 31/16; A61K 45/06

(57) ABSTRACT

Mitochondrially targeted antioxidants are used to prevent and treat severe inflammatory conditions including viral infection (such as COVID-19), systemic shock, trauma, burns, surgery associated with significant tissue damage, toxic damage, and damage associated with autoimmune reactions.

### CROSS-REFERENCE TO RELATED APPLICATIONS

This application claims priority to U.S. Provisional Application No. 63/004,893, filed 3 Apr. 2020, and to U.S. Provisional Application No. 63/011,920, filed 17 Apr. 2020, both of which are incorporated by reference herein in their entirety.

### BACKGROUND

Conditions such as severe viral infections (including COVID-19), systemic inflammatory response syndrome, trauma or surgery accompanied with substantial tissue damage, cold or heat shock, and toxic shock lack adequate treatment methods. There is a need for improved methods for preventing and treating such conditions, including the severe inflammatory reactions and organ and tissue damage associated therewith.

### SUMMARY

The present technology provides methods of using mitochondrially-targeted antioxidants to aid in the prevention and treatment of patients in critical conditions associated with severe or systemic inflammation. Such conditions can be caused, for example, by severe viral or bacterial infections, systemic inflammatory response syndrome of different origin, trauma or surgery accompanied with substantial tissue damage, cold or heat shock, toxic shock, autoimmune conditions, anaphylactic shock, and other pathologies associated with severe or systemic inflammation.

The technology can be further summarized by the following list of features.

1. A method to aid in treating or preventing an inflammatory condition in a subject in need thereof, the method comprising administration of a mitochondrially-targeted antioxidant of formula I (SkQ)

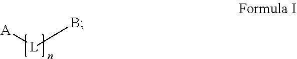

wherein A is an effector moiety/antioxidant optionally having the following structure:

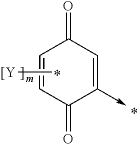

and/or a reduced form thereof, wherein m is an integer from 1 to 3; each Y is independently selected from the group consisting of C₁₋₆ alkyl, C₁₋₆ alkoxy, or two adjacent Y groups, together with carbon atoms to which they are attached, forming the following structure:

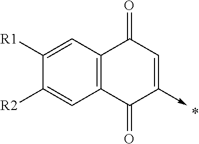

and/or a reduced form thereof, wherein R1 and R2 may be the same or different and are each independently C₁₋₆ alkyl or C₁₋₆ alkoxy;

wherein L is a linker group, comprising:
a) straight or branched hydrocarbon chain which can be optionally substituted by one or more substituents and optionally contains one or more double or triple bonds; and/or
b) a natural isoprene chain;

wherein n is integer from 1 to 40;

wherein B is a mitochondria-targeting group comprising a lipophilic cation and a pharmacologically acceptable anion;

and/or solvates, salts, isomers, or prodrugs thereof.
2. The method of feature 1 wherein SkQ is compound SkQ1 shown below, in its reduced or oxidized form:

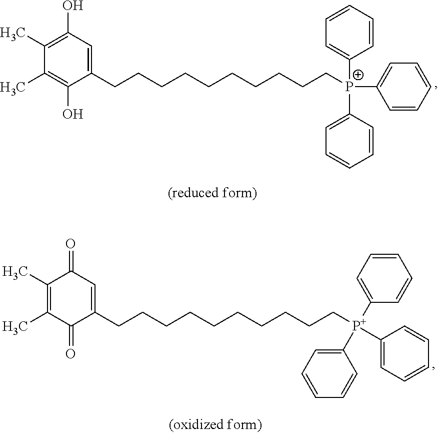 [SMILES: Cc1c(O)cc(CCCCCCCCCC[P+](c2ccccc2)(c2ccccc2)c2ccccc2)c(O)c1C ; CC1=C(C)C(=O)C(CCCCCCCCCC[P+](c2ccccc2)(c2ccccc2)c2ccccc2)=CC1=O]

or a combination thereof.
3. The method of feature 1, wherein SkQ is a compound selected from the group consisting of the following compounds:

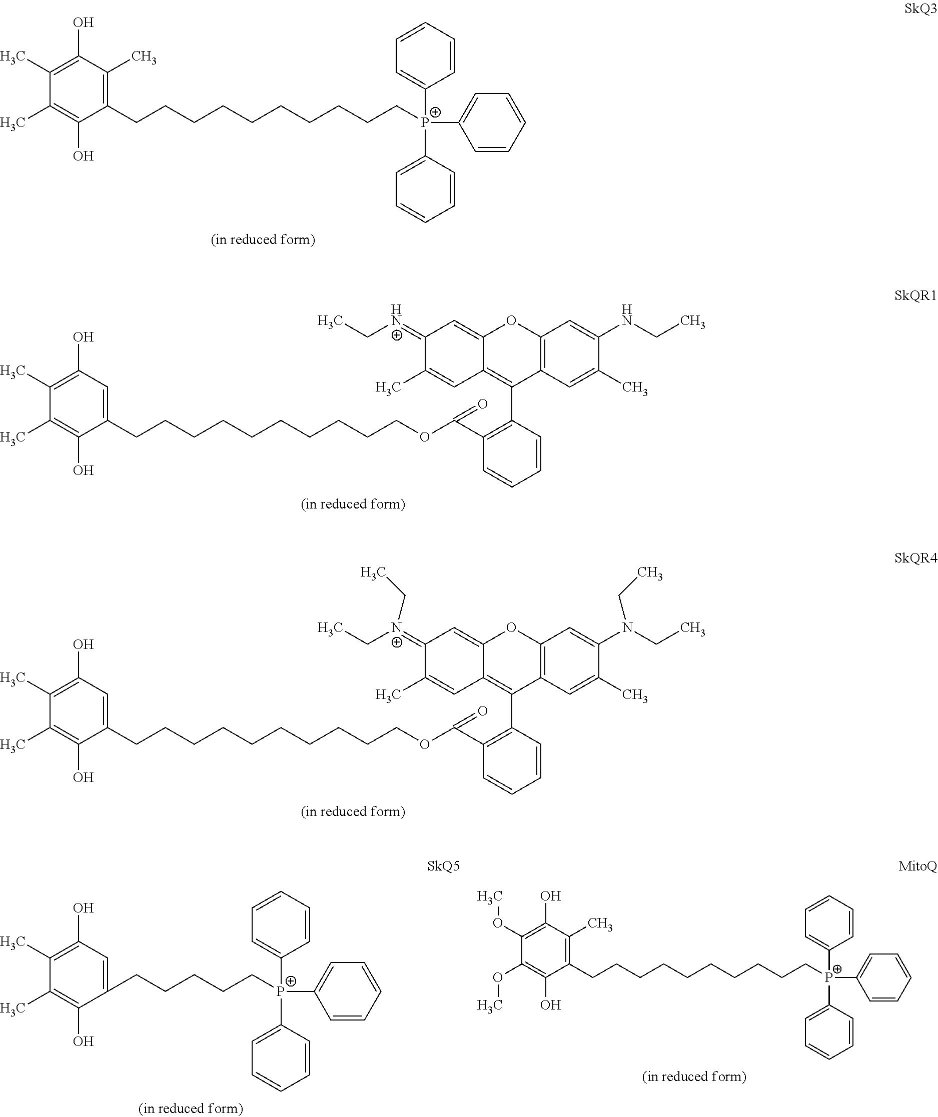

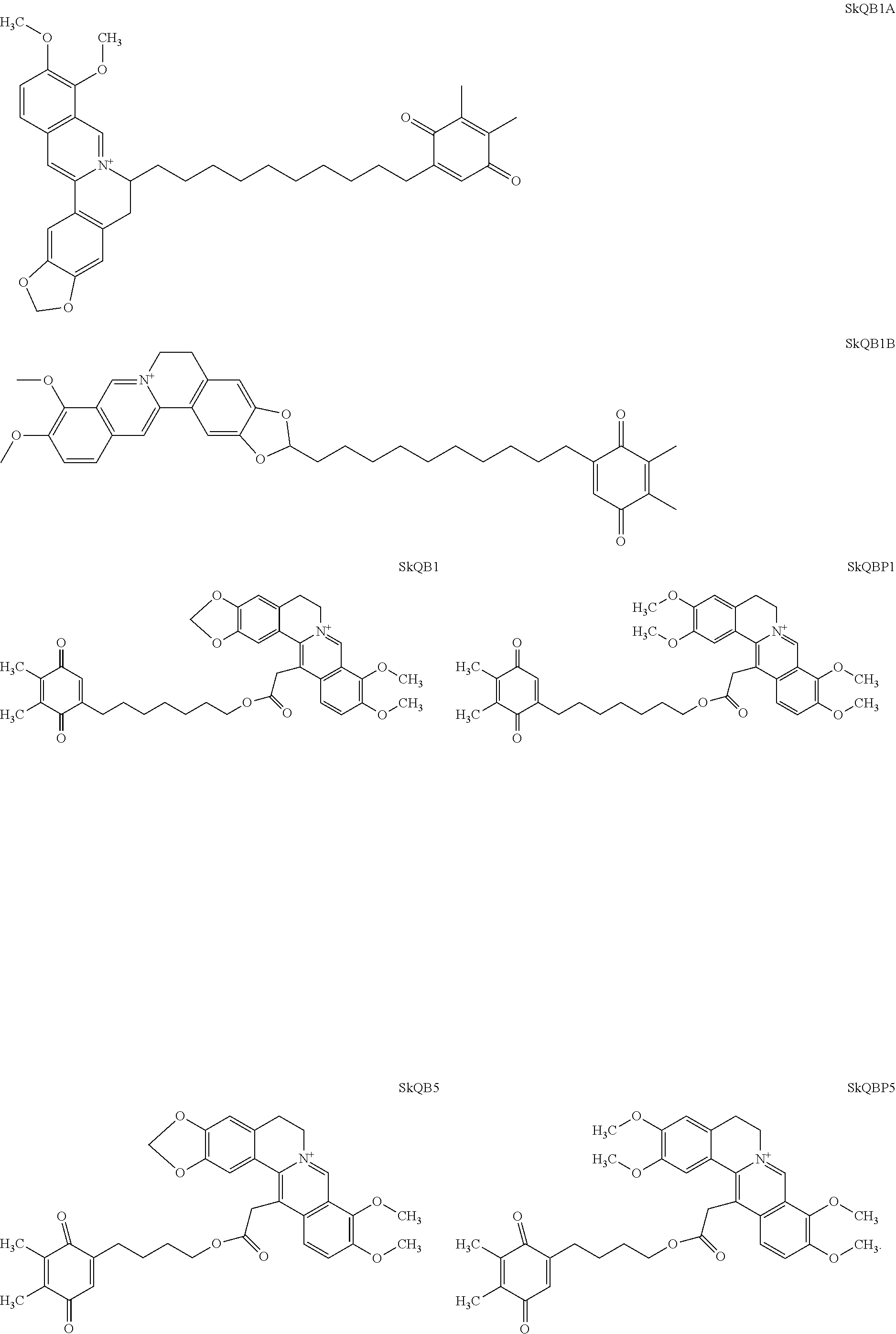

4. The method of any of the preceding features, wherein the inflammatory condition is associated with infection, COVID-19, sepsis or septic shock, cytokine storm or SIRS, vascular endothelial damage, activation of vascular endothelial cells, PAMPS, DAMPS, cold shock, heat shock, toxic shock, burn trauma, surgery, autoimmune disease, anaphylaxis, cancer, ischemic disease, or presence of CIRP and/or HMGB1 in the blood of the subject.
5. The method of feature 4, wherein the infection is bacterial infection, fungal infection, or viral infection.
6. The method of feature 5, wherein the viral infection is infection by an influenza virus or a corona virus, such as SARS-CoV-2.
7. The method of any of the preceding features, wherein SkQ is administered by intravenous injection or infusion, subcutaneous injection, or a slow release formulation or device.
8. The method of any of the preceding features, wherein the method prevents deconstruction of cell-to-cell contacts between endothelial cells of the subject.
9. The method of any of the preceding features, further comprising administering an additional therapeutic agent.
10. The method of any of the preceding features, wherein the method prevents the subject from contracting COVID-19.
11. A kit comprising mitochondrially-targeted antioxidant SkQ of formula I below and instructions for performing the method of any of the preceding features:

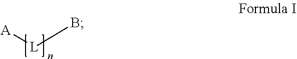

wherein A is an effector moiety/antioxidant optionally having the following structure:

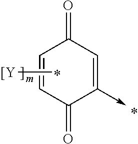

and/or a reduced form thereof, wherein m is an integer from 1 to 3; each Y is independently selected from the group consisting of C₁₋₆ alkyl, C₁₋₆ alkoxy, or two adjacent Y groups, together with carbon atoms to which they are attached, forming the following structure:

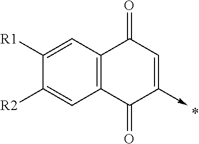

and/or a reduced form thereof, wherein R1 and R2 may be the same or different and are each independently C₁₋₆ alkyl or C₁₋₆ alkoxy;

wherein L is a linker group, comprising:
a) straight or branched hydrocarbon chain which can be optionally substituted by one or more substituents and optionally contains one or more double or triple bonds; and/or
b) a natural isoprene chain;

wherein n is integer from 1 to 40;

wherein B is a mitochondria-targeting group comprising a lipophilic cation and a pharmacologically acceptable anion;

and/or solvates, salts, isomers, or prodrugs thereof.
12. A composition for use in treating or preventing an inflammatory condition in a subject in need thereof, the composition comprising a mitochondrially-targeted antioxidant of formula I (SkQ)

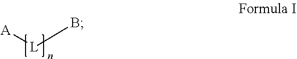

wherein A is an effector moiety/antioxidant optionally having the following structure:

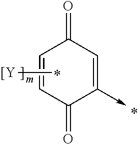

and/or a reduced form thereof, wherein m is an integer from 1 to 3; each Y is independently selected from the group consisting of C₁₋₆ alkyl, C₁₋₆ alkoxy, or two adjacent Y groups, together with carbon atoms to which they are attached, forming the following structure:

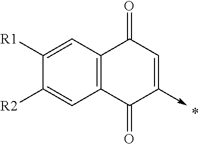

and/or a reduced form thereof, wherein R1 and R2 may be the same or different and are each independently C₁₋₆ alkyl or C₁₋₆ alkoxy;

wherein L is a linker group, comprising:
a) straight or branched hydrocarbon chain which can be optionally substituted by one or more substituents and optionally contains one or more double or triple bonds; and/or
b) a natural isoprene chain;

wherein n is integer from 1 to 40;

wherein B is a mitochondria-targeting group comprising a lipophilic cation and a pharmacologically acceptable anion;

and/or solvates, salts, isomers, or prodrugs thereof.
13. The composition of feature 12 wherein SkQ is compound SkQ1 shown below, in its reduced or oxidized form:

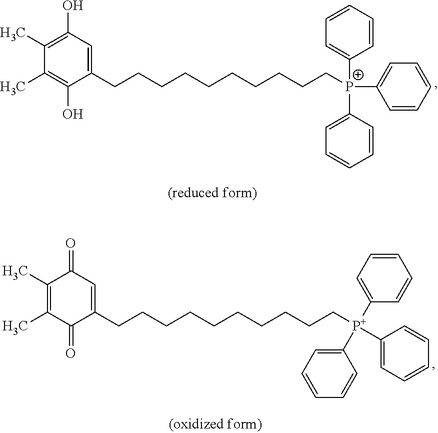 [SMILES: Cc1c(O)cc(CCCCCCCCCC[P+](c2ccccc2)(c2ccccc2)c2ccccc2)c(O)c1C ; CC1=C(C)C(=O)C(CCCCCCCCCC[P+](c2ccccc2)(c2ccccc2)c2ccccc2)=CC1=O]

or a combination thereof.
14. The composition of feature 12, wherein SkQ is a compound selected from the group consisting of the following compounds:

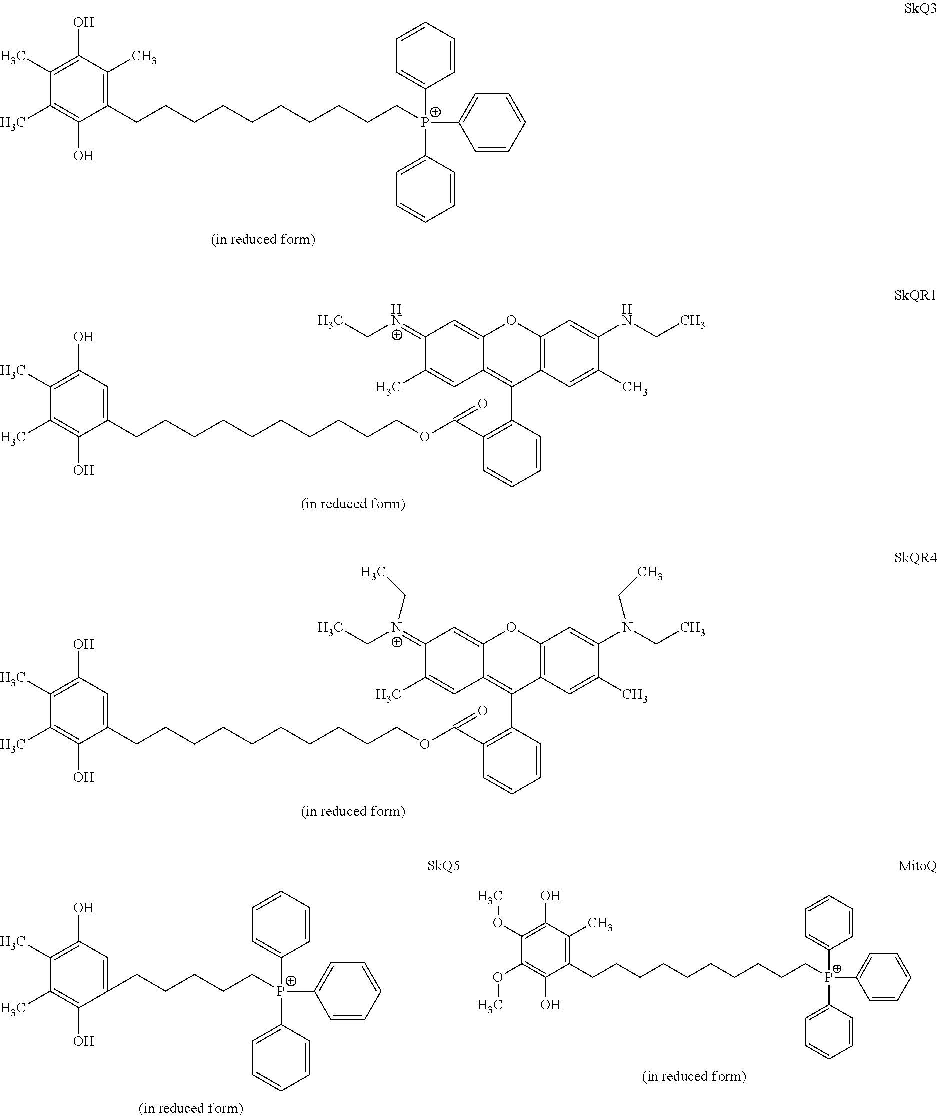

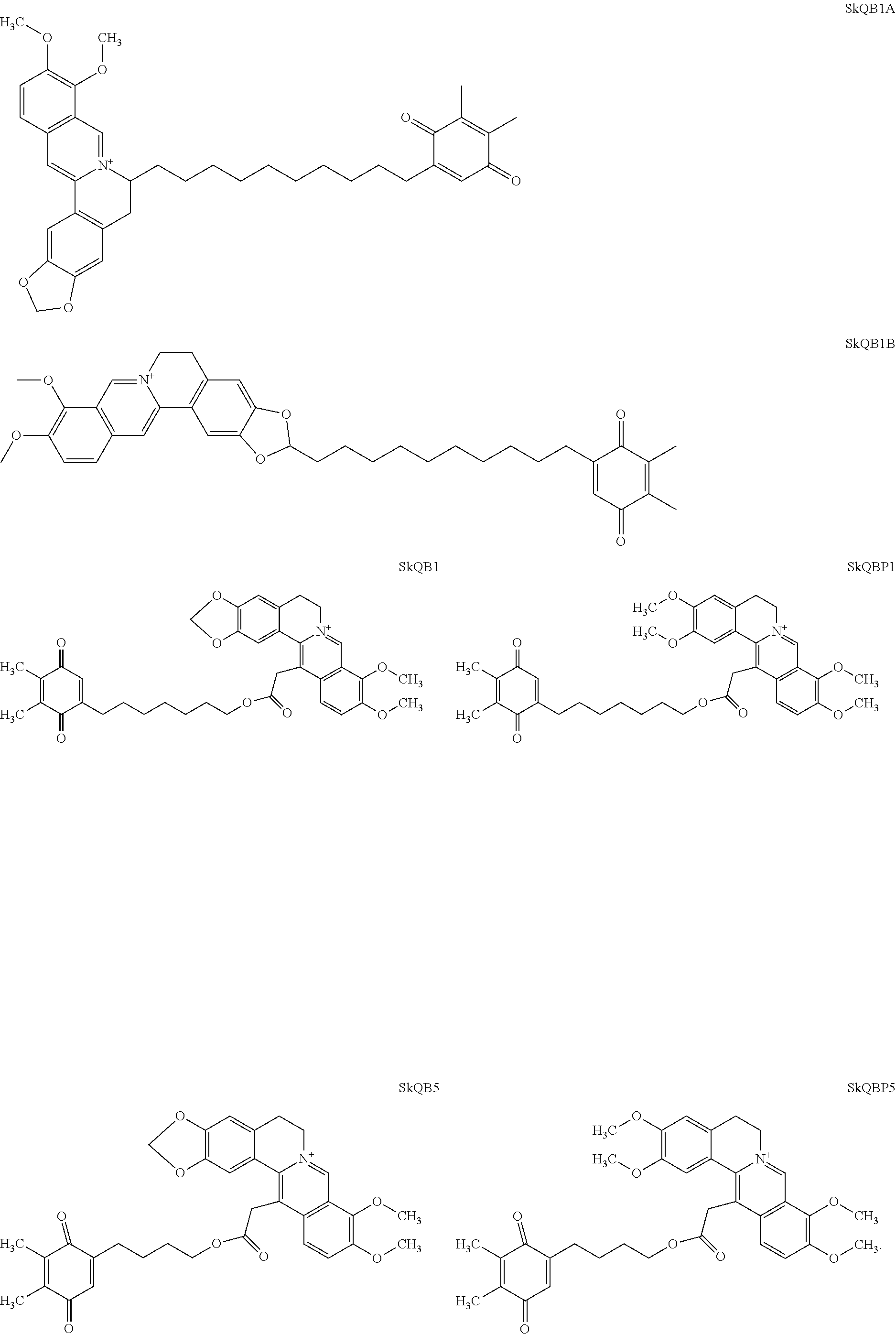

15. The composition of any of features 12-14, wherein the inflammatory condition is associated with infection, COVID-19, sepsis or septic shock, cytokine storm or SIRS, vascular endothelial damage, activation of vascular endothelial cells, PAMPS, DAMPS, cold shock, heat shock, toxic shock, burn trauma, surgery, autoimmune disease, anaphylaxis, cancer, ischemic disease, or presence of CIRP and/or HMGB1 in the blood of the subject.
16. The method of feature 15, wherein the infection is bacterial infection, fungal infection, or viral infection.
17. The method of feature 16, wherein the viral infection is infection by an influenza virus or a corona virus, such as SARS-CoV-2.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of or” “consisting essentially of”.

### BRIEF DESCRIPTION OF THE DRAWINGS

FIGS. 1A-1D show results of LCMS/MS measurements of SkQ1 concentration over time in blood samples from SPF category Wistar rats. FIG. 1A shows SkQ1 blood concentrations over time after SkQ1 was administered by intravenously injection. FIG. 1B shows blood concentrations after SkQ1 was administered by intraperitoneal injection. FIG. 1C shows blood concentrations after SkQ1 was administered by subcutaneous injection. FIG. 1D at left shows comparative LCMS/MS area under the curve (AUC) data for intravenous (i/v), intraperitoneal (i/p), and subcutaneous (i/c) injection. At the right side of FIG. 1D the relative bioavailability is plotted, normalized using i/v as 100%.

FIG. 2 shows plots of prolonged SkQ1 concentration in the blood versus time, with administration using slow-release pump containers introduced subcutaneously into Wistar rats.

FIG. 3 shows western blot results of release of an endothelial marker (vascular endothelial cadherin) after cell cultures of human endothelial cells were exposed to serum from blood samples taken from patients with sepsis. The cell cultures were pretreated with different the indicated amounts of SkQ1, then treated with 5-10% sepsis derived serum, after which a 1 hour VE-cadherin study was performed (wee Example 2). Glyceraldehyde-3-phosphate dehydrogenase (GAPDH) control is shown at bottom.

FIG. 4 shows fluorescence microscopy results after cell cultures of human endothelial cells were exposed to serum derived from patients (age 18-75 years) with diagnosed SIRS. The cell cultures were pretreated with different amounts of SkQ1, treated with 5-10% SIRS serum. Conditions for Control, SIRS serum, SIRS serum+2 nM SkQ1, SIRS serum+20 nM SKQ1, and SIRS serum+200 nm SkQ1 versus Actin, VE-cadherin, and nucleus were as described in Example 2.

FIG. 5 shows the effect of SkQ1 in treating mice infected with influenza virus. The percentage of animals in critical condition (balb\c mice, 10-12 g) is shown after inoculation via nasal injection with H5N1 influenza virus (strain chicken/Kurgan/5/2005). Mice were treated by i/p injection of placebo (n=11), SkQ1 333 nmoles/kg (n=9), SkQ1 1000 nmoles/kg (n=9), or SkQ1 3000 nmoles/kg (n=11). Each animal was treated by i/p injection of 450 μl of saline containing the indicated dosage of SkQ1 or placebo (see Example 3).

FIG. 6 shows % survival versus days after injection with influenza virus (same conditions as FIG. 5).

FIG. 7 shows survival curves after injection of a suspension of isolated liver mitochondria into the tail vein of mice, with the indicated treatments. SkQ1, vehicle, or C12 TPP was injected for five days before mitochondria injection and also for five days after the injection.

FIG. 8 shows survival curves after sharp cold shock of mice induced by exposing the animals to −20° C. for 90 minutes. The treatment conditions were same as for FIG. 7,

FIG. 9 shows survival curves after treatment of mice with a toxic, high concentrations of C12 TPP with and without pretreatment with SkQ1.

FIG. 10 shows survival curves after injection of a suspension of isolated liver mitochondria into the tail vein of mice, with or without treatment with SkQ1 one minute after the mitochondria injection.

FIG. 11 shows the survival rate of mice exposed to shock caused by 90 minutes −20° C. with and without immediate treatment with SkQ1 (see Example 6).

FIG. 12 shows survival curves for mice exposed to a toxic concentration of C12 TPP (34 μmol/kg) with or without treatment with SkQ1 (1.5 μmol/kg) injected at 4.5 hours before C12 TPP injection and 0.5 hours after C12 TPP injection.

FIG. 13 shows survival curves for mice exposed to a toxic concentration of C12 TPP (42 μmol/kg) with or without SkQ1 (1.5 μmol/kg) injected 1 hour after administration of C12 TPP.

FIG. 14A shows TNFα concentration in the blood of mice as a function of time after injection of liver mitochondria with or without SkQ1 injection FIG. 14B shows IL-6 concentration in the blood of mice as a function of time after injection of liver mitochondria either with or without SkQ1 injection.

FIG. 15A shows TNFα concentration in the blood of mice as a function of time after cold shock with SkQ1 or vehicle injection. FIG. 15B shows IL-6 concentration in the blood of mice as a function of time after cold shock with SkQ1 or vehicle injection.

### DETAILED DESCRIPTION

It is known that significant health decline or even death of patients in various critical conditions often occurs due to dangerous responses of the immune system activated in the body of a patient by a trigger, which can be a disease or other pathological condition. Examples of such triggers are severe infections such as viral infections (including COVID-19, which is infection with SARS-CoV-2 virus), bacterial or fungal infections (including sepsis), trauma, cold or heat shock (including burns), tissue damage resulting from surgery, damage due to toxic substances, autoimmune reactions, and anaphylaxis or severe allergic reactions.

An example of a dangerous outcome that is common to many of the above mentioned conditions is systemic inflammation response syndrome (SIRS). SIRS is often associated with the development and progression of acute respiratory distress syndrome (ARDS). Amid the COVID-19 pandemic, hospitals have experienced overburdening demand for treatment of patients in critical condition. Critically ill patients with COVID-19 often experience respiratory failure and are put on ventilators, while receiving specialized care for complicating critical conditions. Most severe cases of COVID-19 result in ARDS, which is a life-threatening lung condition that can be caused by a virus or a wide range of pathogens. Complications of COVID-19-related ARDS can include cardiac injury including cardiomyopathy, pericarditis, pericardial effusion, arrhythmia, and sudden cardiac death, acute kidney injury (AKI), liver damage (elevated liver enzymes), sepsis, and shock. A critical condition known as multiple organ dysfunction syndrome (MODS) can quickly arise.

The present technology provides methods to aid in treatment or prevention a critical condition associated with severe and/or systemic inflammation; it does not include methods for treatment or prevention of local inflammation of a minor nature. The technology can be used to aid in the treatment or prevention of a lethal or life threatening condition related to inflammation within the body of a subject, regardless of the cause. The methods also can be used to reduce risk of developing such a condition in a subject who has a risk of developing such a condition. The methods include administering to a subject in need thereof (i.e., a subject who has or is considered by a medical professional to be likely to develop a condition associated with severe inflammation) a mitochondrially targeted antioxidant of the SkQ type. Such compounds are of general Formula I. These compounds are capable of protecting a subject from developing conditions associated with severe inflammation, and also are capable of treating, or aiding in the treatment of such conditions.

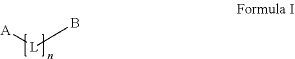

wherein A is an effector moiety, which is preferably an antioxidant, optionally having the following structure:

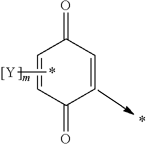

and/or a reduced form thereof (having, for example, one or more ═O reduced to —OH), wherein m is an integer from 1 to 3; each Y is independently selected from the group consisting of: C₁₋₆ alkyl (lower alkyl), C₁₋₆ alkoxy (lower alkoxy); or two adjacent Y groups, together with carbon atoms to which they are attached, form a following structure:

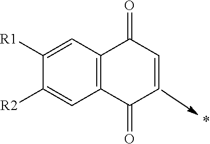

and/or a reduced form thereof;
wherein R1 and R2 may be the same or different and are each independently C₁₋₆ alkyl or C₁₋₆ alkoxy;
L is a linker group, comprising:
a) straight or branched hydrocarbon chain which can be optionally substituted by one or more substituents and optionally contains one or more double or triple bonds; and/or
b) a natural isoprene chain;
n is integer from 1 to 40;
B is a mitochondria targeting group comprising a lipophilic cation and a pharmacologically acceptable anion;
and/or solvates, salts, isomers or prodrugs thereof.

As used herein, an isoprene chain or a natural isoprene chain refers to—[CH₂C(Me)=CHCH₂]e- or —[CH₂C(Me)=CHCH₂]e-OH (one terminal —OH), wherein e is 1, 2, 3, 4, 5, 6, 7, 8, 9, 10; or wherein e is in a range defined by any two members of the group; or wherein e is in the range from 1-40.

Examples of mitochondrially targeted antioxidants of formula (I) are:

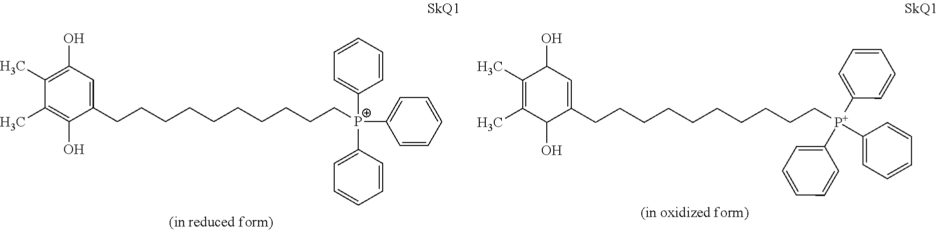 [SMILES: Cc1c(O)cc(CCCCCCCCCC[P+](c2ccccc2)(c2ccccc2)c2ccccc2)c(O)c1C ; CC1=C(C)C(O)C(CCCCCCCCCC[P+](c2ccccc2)(c2ccccc2)c2ccccc2)=CC1O]

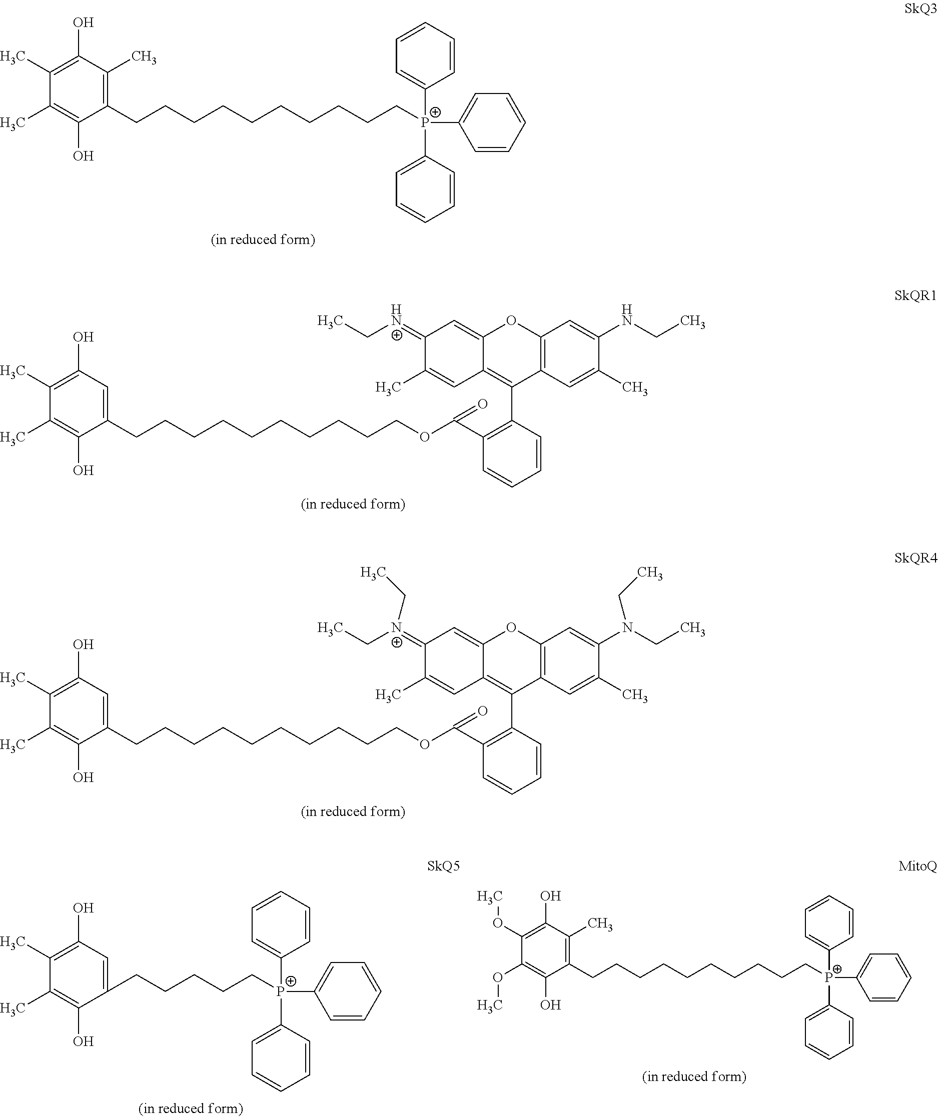

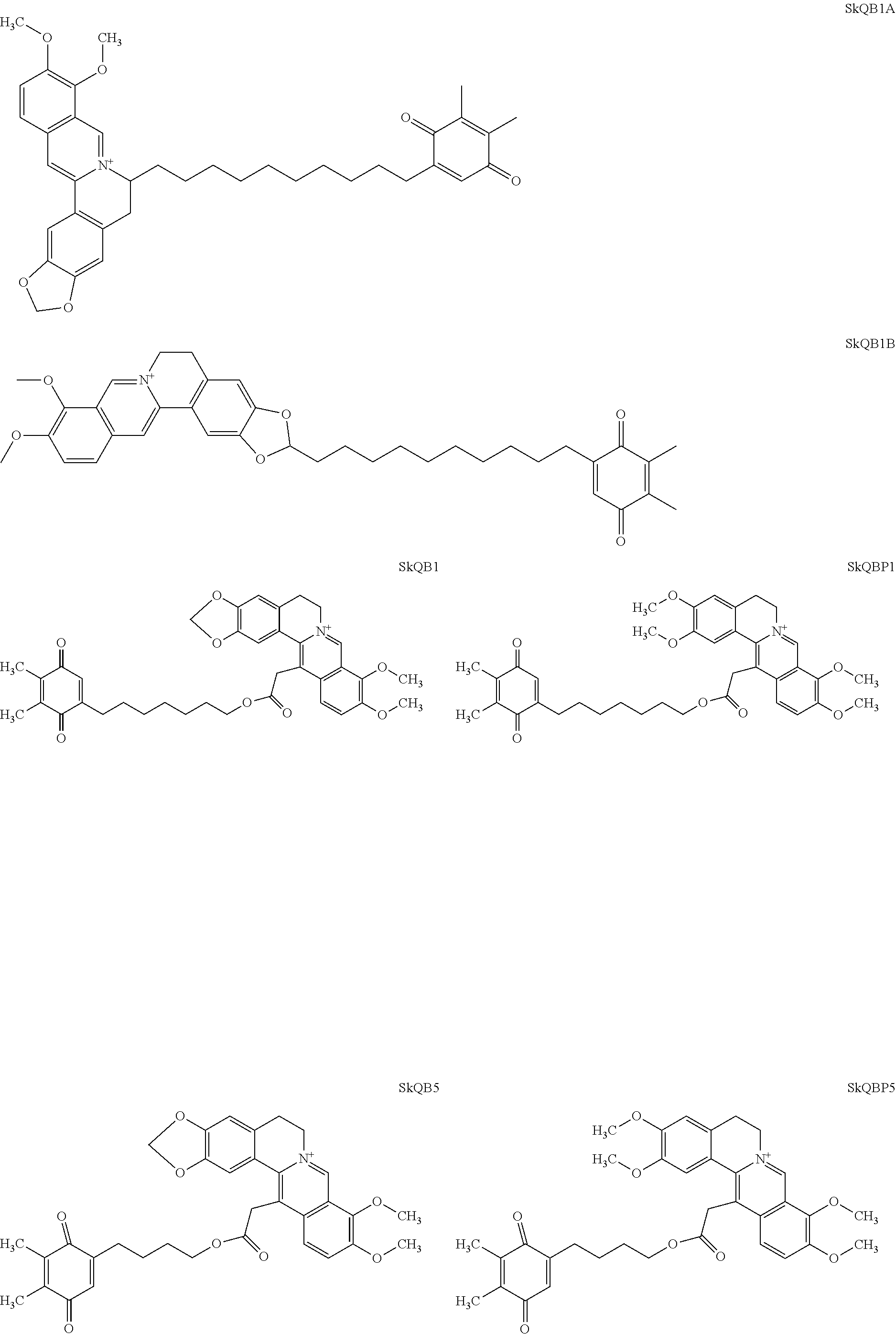

Further SkQ compounds for use in the methods disclosed herein can be derived from SkQ1 or any of the above compounds, for example, by removal of a methyl group from the effector moiety, A. Thus, any of the methods can include administration of a mitochondrially targeted antioxidant SkQ such as the following (in either reduced or oxidized form):

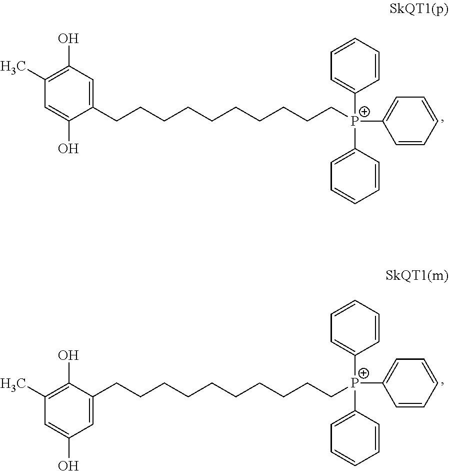 [SMILES: Cc1cc(O)c(CCCCCCCCCC[P+](c2ccccc2)(c2ccccc2)c2ccccc2)cc1O ; Cc1cc(O)cc(CCCCCCCCCC[P+](c2ccccc2)(c2ccccc2)c2ccccc2)c1O]

or a combination thereof.

All of above compounds (SkQ) can be in either reduced or oxidized forms, or a mixture thereof. The reduced form can have, for example, one or more ═O reduced to —OH, and the oxidized form can have an oxidized quinone moiety (as it is shown for SkQ1 above).

Each variant of SkQ compounds can be used in a form of salt with a pharmaceutically acceptable anion such as chloride, bromide, sulfate, phosphate, mesylate, citrate, or acetate. “Neutral” forms of the compounds may be recovered by contacting a salt form with base or acid, with release of the original compound by the traditional way and isolation by, for example, precipitation, elution from a column, or extraction. The original form of the compound can differ from salt forms in certain physical properties, for example, solubility in polar solvents.

The SkQ compounds, either alone or in combination, can be provided in a purity range from about 80% to about 100%, from about 90% to about 100%, from about 95% to about 100%, from about 99% to about 100%, from about 99.5% to about 100%, from about 99.9% to about 100%. Examples of measuring purity are use of a diode array detector to monitor eluate from a UHPLC column and integrating the main absorbance peak (SkQ) along with any impurity peaks. In an example, the SkQ compounds disclosed herein can be formulated into prodrugs by formation of ester between an —OH group on the SkQ compound and a suitable transformation group, such as a glycyl ester, an amino ester, or a polymer ester.

One of the routes of administration of these compounds (SkQ) to patients is i/v injection. Experimental results indicate that reasonable dosage range for humans can be from about 1 mg to about 22 mg of SkQ1 per day (Example 4). The dosage range can optionally be divided into several injections (for example, 1 injection each 12 hours). A dosage range for humans can be in the range from about 1 mg to about 5 mg of SkQ1 per day, from about 1 mg to about 10 mg of SkQ1 per day, from about 1 mg to about 15 mg of SkQ1 per day, from about 1 mg to about 20 mg of SkQ1 per day, or from about 1 mg to about 22 mg of SkQ1 per day. With less bioavailability, depending upon the route of administration, the upper limit can be higher. Another way of administration can be gradual intravenous infusion of SkQ (Example 1). SkQ also can be administered by subcutaneous or intraperitoneal injection in the above described dosage range. Formulation of i/v formulations, subcutaneous, or intraperitoneal formulations can include adjustment of the formulation osmolality to match either i/v, subcutaneous, or intraperitoneal conditions. Optionally excipients, preservatives, other active agents (e.g., combination or cocktail therapy) can be included.

It is known that ARDS is tightly linked to systemic inflammation that occurs in patients with massive viral infection. One of the key elements of systemic inflammation is so called “cytokine storm” that affects primarily vascular endothelium. SIRS or other inflammation associated mechanisms often lead to the damage of vascular endothelium. The health decline and risk of death in many critical conditions can occur due to the damage to endothelium. One of the uses of the present technology is to aid in the treatment or prevention of damage to vascular endothelium associated with a severe or systemic inflammatory condition by protection of vascular endothelium with SkQ (see Example 2).

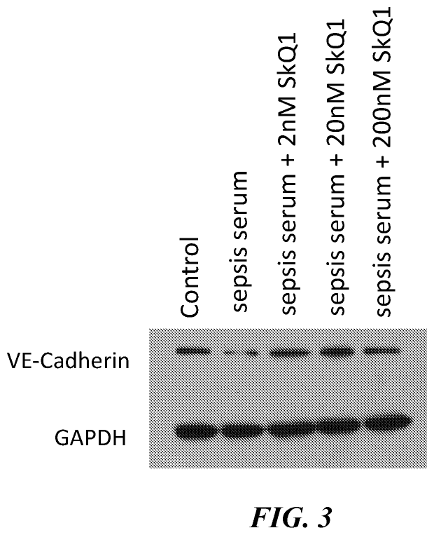

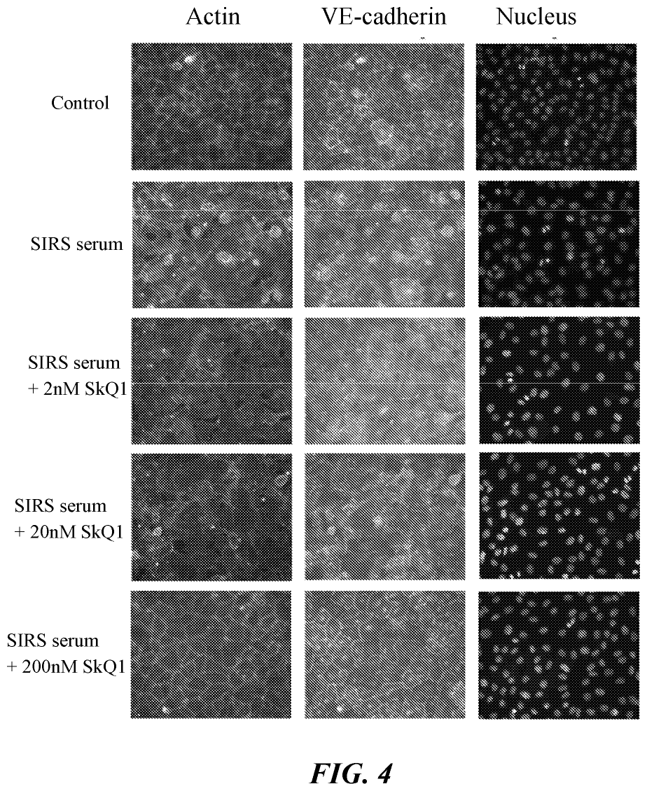

The mitochondrially targeted antioxidants disclosed herein can prevent deconstruction of cell-to-cell contacts between human vascular endothelial cells in culture when these cells are treated with blood serum samples from patients with SIRS or sepsis. Results of a VE-cadherin western blot are presented on FIG. 3, wherein a cell culture of human endothelial cells is pretreated with different dosages of SkQ1, then exposed to serum from patients diagnosed with sepsis. At the right of FIG. 3, the VE-cadherin, with exposure to 200 nM SkQ1 and serum, is about equal intensity to the control at left. In FIG. 4, blood samples are taken from patients with diagnosed SIRS, with the patients age 18-75 years. A cell culture of human endothelial cells is pretreated with different dosages of SkQ1, then treated with 5-10% serum from the SIRS blood samples. At the top of FIG. 4, the VE-cadherin (fluorescence microscopy) c resembles the VE-cadherin at the bottom of FIG. 4 (SIRS serum+200 nM SkQ1). Actin and nucleus visualizations in FIG. 4 correspond with VE-cadherin observations.

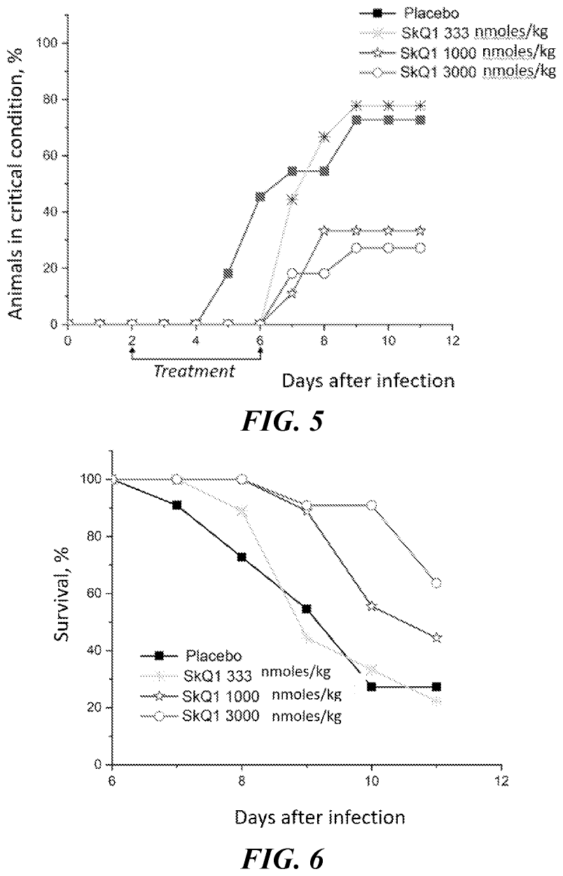

In order to demonstrate that various critical conditions can be treated or prevented by SkQ, experiments are performed on several animal models of such critical conditions. Experiments were selected to model critical needs. Example 3 presents a model of severe viral infection. Nasal injection of influenza virus H5N1 induces most mice into critical condition in the placebo condition (450 μL saline) shown in FIG. 5. Intraperitoneal administration of SkQ1 (1000 nmoles/kg and 3000 nmoles/kg) protects against the critical condition. The percent survival is plotted in FIG. 6.

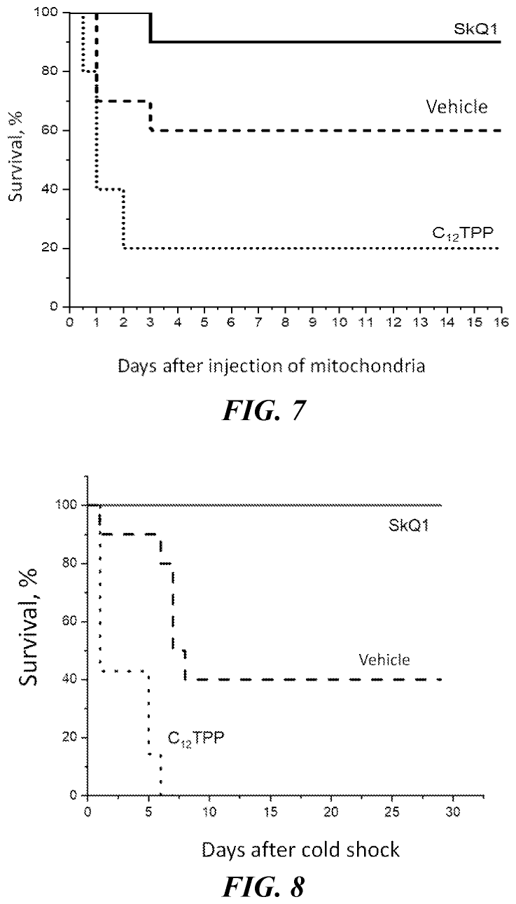

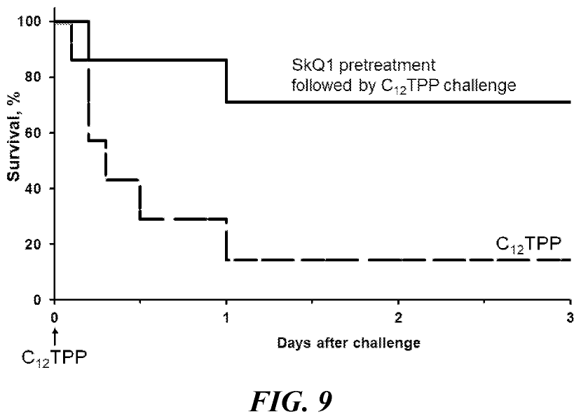

In Example 5, severe shock is modeled on mice using intravenous injection of mitochondria, cooling (−20° C.), or poisoning with C12 TPP. In the intravenous injection of mitochondria and cooling models, pretreatment of and post-treatment of the mice with SkQ1 is effective for high percent survival outcomes. FIG. 7 shows the survival percentage for the intravenous injection of mitochondria, and FIG. 8 shows the survival percentage for the cold shock. In the C12 TPP model (FIG. 9), conditions without the SkQ1 pretreatment show a 90% mortality within 1 day, while SkQ1 pretreatment prevents mortality, in a same manner as it prevents mortality caused by i/v injection of mitochondria or cold shock. Example 6 demonstrates the effectiveness of immediate (about one minute) administration of the mitochondrially targeted antioxidants. Example 7 further explores the effects of SkQ1 on cytokines TNFa and IL-6. Thus, an example of the technology is the use of an SkQ compound for prevention or treatment of human patients in critical conditions corresponding to the animal models demonstrated.

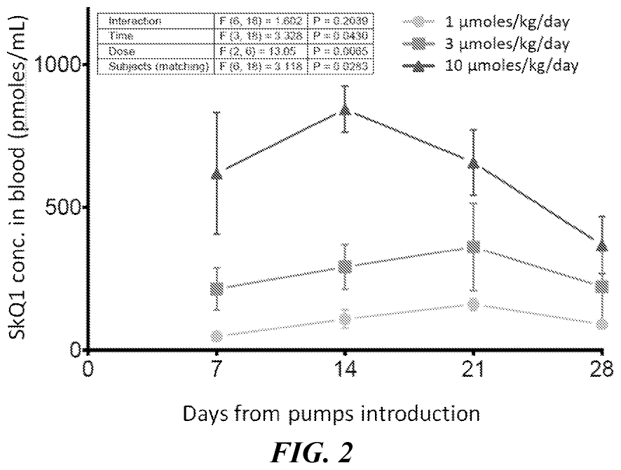

For example, treating a patient with a mitochondrially targeted antioxidant disclosed herein can be performed immediately upon or after exposure to a trigger of a critical condition, within about 30 seconds after, within about 1 minute after, within about 5 minutes after, within about 30 minutes after, within about an hour after, within about 2 hours after, within about 5 hours after, within about 1 day after, or within about 3 days after. In an example, an emergency or lifesaving treatment can include treatment immediately upon or after exposure to a trigger of a critical condition. Examples of pre-treatments can include treating a patient about 10 days, about 5 days, about 1 day, about 12 hours, about 6 hours, or about 1 hour before a critical condition. Examples of post-treatments can include treating a patient about 10 days, about 5 days, about 1 day, about 12 hours, about 6 hours, or about 1 hour after a critical condition. As illustrated in FIG. 2, a continuous infusion of the therapies and compositions can be utilized.

The inventors have demonstrated that lethal effects of different forms of shock, each associated with severe, systemic inflammation, can be prevented or treated with the administration of an SkQ compound. For example, the lethal effects of (a) the injection of mitochondria into the blood of mice, (b) hypothermia, and (c) injection of C12 TPP at toxic levels, can be prevented by administration of the mitochondrially targeted antioxidant SkQ1. These results indicate that SkQ compounds can prevent critical conditions triggered by very different inflammation-associated conditions in the mammalian organism. These conditions apparently share underlying mechanisms such as cytokines, signal messengers, and possibly proteins. Without intending to limit the technology to any particular mechanism, it is believed that at least two candidates for such common underlying mechanisms are: cold inducible RNA-binding protein (CIRP) and high-mobility group box 1 protein (HMGB1). Therefore, another example of the technology is a method of prevention or treatment of an inflammatory condition, the method including administration of an effective and safe dosage of SkQ to a patient having a condition triggered by a mechanism involving CIRP, or HMGB1, or another messenger that directly or indirectly interacts with toll-like receptors.

Another example of the technology includes administration of SkQ to a patient experiencing a condition, or who is in risk of developing a condition, characterized by severe or systemic inflammation. Such treatment includes (but not limited to) subcutaneous, intraperitoneal or intravenous administration of SkQ. The administration can be through an emergency auto-injector or emergency pen. Such conditions include viral infection, bacterial infection (including sepsis), fungal infection, autoimmune disease, SIRS, vascular endothelial damage, thrombosis, trauma, cold or heat shock (including burns), surgery associated with significant tissue damage, and damage from exposure to a toxic substance. A related example is protection of the patient's vascular endothelial cells from apoptosis, necrosis and/or inflammatory activation.

Absorption through skin (topical) formulations or by administration of microparticles or nanoparticles with any of the above described routes is considered within the present technology. Other examples of formulations for administration of SkQ are inhalation formulations including inhalation of particles or mist, and oral formulation.

A method of making a medicine or a formulation for protection and treatment of patients in critical conditions includes the steps of: providing a vehicle, and contacting the vehicle with a mitochondrially targeted antioxidant of SkQ type (a compound of general formula I). The compound of general formula I can be provided as a salt, for example, a pharmaceutically acceptable salt. Examples include, but are not limited to, citrate, hexanoate, hexylresorcinate, hydrabamine, hydrobromide, hydrochloride, hydroiodide, 2-hydroxy-ethanesulfonate, hydroxynaphtoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, clavulanate, cyclopentane propionate, digluconate, dihydrochloride, dodecylsulfate, edetate, edisilate, estolate, esilate, ethanesulfonate, formiate, fumarate, gluceptate, glucoheptonate, gluconate, glutamate, glycerophosphate, glycolylarsanilate, hemisulfate, heptanoate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, theoclate, tosylate, triethiodide, undecanoate, valerate, acetate, adipate, alginate, ascorbate, aspartate, benzenesulfonate, benzoate, bicarbonate, bisulfate, bitartrate, borate, bromide, butyrate, calcium salt of ethylene diaminetetraacetic acid (edetate), camphorate, camphorsulfonate, camsilate, carbonate, and chloride. The vehicle can be gas, solid, or liquid. In one example, the medicine or formulation consists of an aqueous vehicle and the compound of general formula I or a salt thereof.

In the making a medicine or a formulation, the compound of general formula I can be provided in a liquid, gas, atomized, or solid form and can optionally include a particle size distribution, measured, for example on a volume, number, or weight basis including a range from a few nanometers to about 1 millimeter. The method can further include adding a pharmaceutically acceptable carrier, or the vehicle can be a pharmaceutically acceptable carrier. The method can include micronization, grinding, sonic mixing or sonication, homogenization, or a combination thereof.

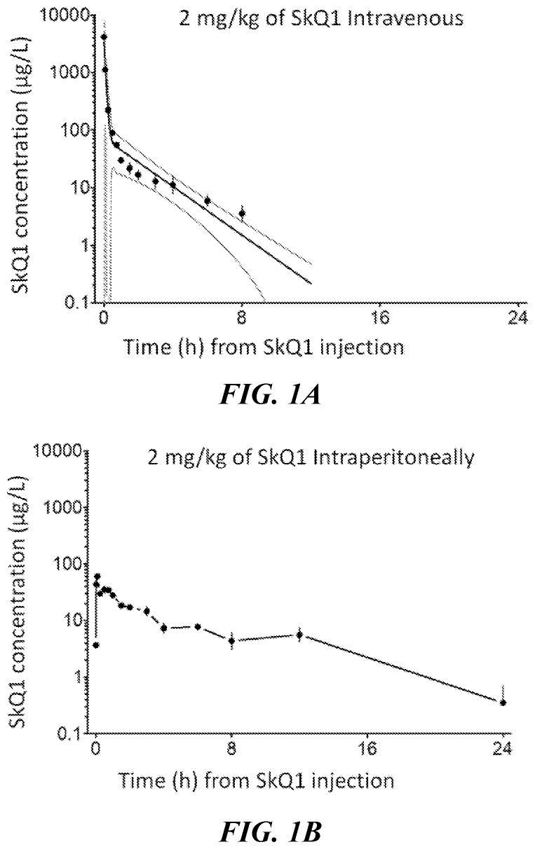

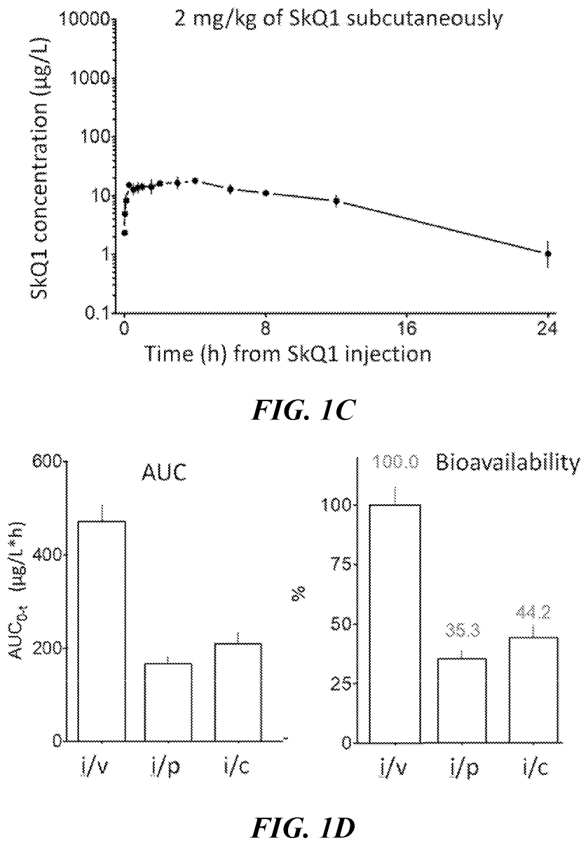

The compound of general formula I can be provided in a therapeutically effective amount. The medicine or a formulation can be provided in a concentrated form, which is later diluted to a therapeutically effective amount. Dilution can be by injection or administration of a concentrated solution or formulation. A therapeutically effective amount can provide a daily dosage for humans in the range from about 1 mg to about 5 mg of the compound of general formula I per day or per every 12 hours, from about 1 mg to about 10 mg of the compound of general formula I per day or per every 12 hours, from about 1 mg to about 15 mg of the compound of general formula I per day or per every 12 hours, from about 1 mg to about 20 mg of the compound of general formula I per day or per every 12 hours, or from about 1 mg to about 22 mg of the compound of general formula I per day or per every 12 hours. As used herein, a safe and effective amount or a safe dosage is about the upper limit of the therapeutically effective amounts described herein. If bioavailability is low, depending on the route of administration, a safe and effective amount or a safe dosage can be adjusted higher to consider low bioavailability, for example, as shown in FIG. 1D.

The method of making a medicine or a formulation can include pH adjustments using any pharmaceutically acceptable acid or base. For example, citrate can used for pH adjustment of subcutaneous formulations because of low skin irritation, and other examples include HCl, maleic acid, tartaric acid, lactic acid, acetic acid, sodium bicarbonate, and sodium phosphate. The pH of the medicine or a formulation can be in the range of about 3.0-9.0, in the range of about 4.0-8.0, in the range of about 5.0-8.0, in the range of about 6.0-8.0, or in the range of about 7.0-8.0, or about 5.0, or about 7.4. In an example, a subcutaneous medicine or formulation can be about pH 5.0. An i/v medicine or formulation can be about pH 7.4. The osmolality of the medicine or formulation can be adjusted by adding, for example, NaCl, KCl, Na/KH₂PO₄, sucrose, ions, or buffers. The osmolality of the medicine or a formulation can be about 300 mOsm/kg, or about 600 mOsm/kg, or in the range from about 200 mOsm/kg to about 700 mOsm/kg, or in the range from about 200 mOsm/kg to about 600 mOsm/kg, or in the range from about 300 mOsm/kg to about 600 mOsm/kg. A hypertonic solution can be provided to reduce the total volume injected, but a higher hypertonicity can cause injection pain, and an upper limit of 600 mOsm/kg can minimize hypertonicity-induced pain.

Procedures of administration can include enteral, such as oral, sublingual and rectal; local, such as transdermal, intradermal and oculodermal; and parenteral. Suitable parenteral procedures of administration include injections, for example, intravenous, intramuscular, subdermal, intraperitoneal, intra-arterial, and other injections, and non-injecting practices, such as vaginal, nasal, rectal, as well as procedures of administration of a pharmaceutical composition as an angioplastic stent coating. The medicine or a formulations can be administered by routes including, for example, intraperitoneal, intravenous, intra-arterial, parenteral, and inhalation.

The method can include providing other ingredients, for example, organic solvents (e.g., ethanol, methanol, DMSO, acetonitrile, propylene glycol(s), N-methyl-2-pyrrolidone, glycofurol, glycerol formal, acetone, tetrahydrofurfuryl alcohol, diglyme, dimethyl isosorbide, and ethyl lactate), other active agents, preservatives, solubility agents, buffers, biologics, cells, chelation agents, and colorants.

SkQ compounds can be used for manufacturing of a medicine for protection and treatment of patients in severe or systemic inflammatory conditions. The compositions and methods disclosed herein can be provided in a kit for emergency treatment of a patient. The kit can be utilized to prevent a severe inflammatory condition from turning to a lethal condition, for example, during transport of a patient to an emergency care facility. For example, the methods and compositions described herein can be provided in the form of an emergency kit for treating hot or cold shock, systemic shock, viral infection, for treating blood vessels to prevent or reverse damage caused by COVID-19, for treating toxic shock or poisoning, shock from severe tissue damage, septic shock, or for prevention of lethal conditions arising from a yet to be specifically diagnosed shock or inflammatory condition in the patient. The kit can include an emergency administration pen including an injectable formulation of SkQ. The kit can include a syringe including an injectable formulation of SkQ. The kit can include SkQ in a penetrable vial or in an ampule, and a syringe can be provided for filling with SkQ.

In another example, a method of protecting a subject suspected of having been exposed to a condition causing a severe or systemic inflammatory condition can include administration of SkQ before, during, or after the exposure. For example, a person entering an area containing or suspected of containing toxins, heat, cold, bacteria, viruses such as influenza, SARS-CoV-2, or other persons infected with a virus or other microbe can receive a prophylactic does of SkQ.

Administration of SkQ compounds, or compositions containing them, can be accomplished, for example, by continuous intravenous infusion of the compound or compositions. Administration can be by a wearable breathing mask infused with a mist, particles, or vapor including SkQ. A wearable device for persons known to be exposed to conditions causing a patient in critical condition, or persons known to be exposed to extreme hot or cold, or any suspected danger to health and safety is also contemplated. Devices including drip, infusion, and implanted catheter devices provide control over the amount and rate of SkQ administered to a patient in need. The devices disclosed herein include fixed ventilators, such as hospital ventilators including SkQ in the inhaled air.

The presently disclosed methods utilizing the mitochondrially targeted antioxidant SkQ compounds can be used to treat inflammation from a variety of sources. Examples include allergic reactions, adverse reactions to chemicals, drugs, or foods, stress, bacterial infections, viral infections, acute respiratory distress syndrome (ARDS), systemic inflammatory response syndrome (SIRS) or sepsis, graft-versus-host disease (GVHD), organ transplantation, tumor lysis syndrome, multiple sclerosis, pancreatitis, and reaction to a vaccine or therapeutic protein, In some cases an inflammatory response can trigger a cytokine storm or cytokine release syndrome (CRS), which can be treated by administering a mitochondrially targeted antioxidant compound as disclosed herein. Examples of viral infections associated with inflammation, which can be treated by administering a mitochondrially targeted antioxidant compound as disclosed herein include, COVID-19, SARS, MERS, influenza, and Ebola.

The compositions and methods of treatment provided by the present technology can provide a therapy to a patient who may have deteriorated to critical condition in a clinical setting or to prevent such occurrence. A patient may exhibit an initial clinical presentation that rapidly becomes a critical condition, for example, or have cough, fever, sore throat, otitis/ear pain, upper respiratory infection, abdominal pain, vomiting, diarrhea with or without vomiting, acute dermatitis or rash, trauma, joint or limb inflammation or pain, or CNS symptoms. Examples of conditions that cause a patient to deteriorate to a critical condition can arise from chronic conditions, such as Alzheimer's disease, arthritis, chronic obstructive pulmonary disease (COPD), depression, diabetes, heart disease, high blood pressure, long-term exposure to toxins or drugs, high cholesterol, obesity, osteoporosis, or stroke.

The present compounds and methods can be used to treat or prevent severe or systemic conditions caused by, for example, acute urticaria, allergic rhinitis, anaphylaxis, animal bite wounds, appendicitis, arthritis (e.g., juvenile rheumatoid arthritis, JRA), asthma or asthma attack, atopic dermatitis, bacteremia-occult, brain tumor, broken bone, bronchitis, burn, cellulitis, cervical adenitis, chlamydia pneumonitis, closed head trauma, colic, common cold, complications during pregnancy (e.g., intrahepatic cholestasis), congenital adrenal hyperplasia, congenital hip dislocation, conjunctivitis, constipation, contact dermatitis, croup, cystic fibrosis, dehydration, diabetic ketoacidosis (DKA), drug reaction, encopresis, acute failure to thrive, febrile convulsions, foreign body aspiration, gastritis, gastroenteritis, gastroesophageal reflux, giardia, headaches (migraine, tension), heart attack, hemolytic/uremic syndrome, hepatitis, hydrocephalas, impetigo/cellulitis, incarcerated hernia, increased intracranial pressure, other infections (e.g., toxic tenosynovitis, septic arthritis, osteomyelitis), inflammatory bowel disease (IBD), acute injuries from child abuse, injury resulting from a fall or an automobile accident, intussusception, Kawasaki's disease, Legg-Calve′-Perthes disease, leukemia/tumors, malignancy, mastoiditis, meningitis, combined microbial (e.g., fungal or bacterial) infections, middle ear effusion, misuse of drugs or medications, monilial skin infections, mononucleosis, nurse maid's elbow, Osgood-Schlatter disease, osteomyelitis, otitis media, ovarian/testicular torsion, pelvic inflammatory disease (PID), peptic ulcer, periorbital/orbital cellulitis, peritonsillar and retropharyngeal abscesses, pertussis, pharyngitis (e.g., strep w, w/o scarlatina, or viral), pneumonia, prions, psychogenic abdominal pain, pyelonephritis, pyloric stenosis, rabies, recurrent otitis media, respiratory infection, rheumatic fever, roseola, scabies, scarlet fever, seborrheic dermatitis, conditions secondary to infections (e.g., strep pharyngitis, otitis), seizure disorders, febrile convulsions, septic arthritis, sickle cell crisis, sinusitis, slipped femoral capital epiphysis, Stevens-Johnson syndrome, strep throat, tendonitis, tetanus, tinea infections, tonsillitis, tuberculosis, unexplained shock, upper respiratory infection (URI), urinary tract infection (UTI), pyelonephritis, vasculitis (e.g. Henoch-Schonlein purpura, HSP), viral exanthems (varicella, measles or rubella, fifth disease), suspected viral illness (unconfirmed or nonspecific), or volvulus/bowel obstruction.

### EXAMPLES

### Example 1. SkQ1 Bioavailability Study: Comparison of Intravenous, Subcutaneous, Intraperitoneal Administration

The bioavailability of SkQ1 was determined after using different methods of administration. SPF category Wistar rats were used. SkQ1 concentration in blood samples was measured using an LCMS/MS method SkQ1 was administered at 2 mg\kg, and blood samples were taken at the indicated time points. Administration was by intravenous injection (FIG. 1A), intraperitoneal injection (FIG. 1B), or subcutaneous injection (FIG. 1C). Total SkQ1 dose was calculated as area under the curve (AUC) as shown at left of FIG. 1D. SkQ1 bioavailability was normalized to that of i/v administration and is shown at the right side of FIG. 1D. SkQ1 bioavailability for intraperitoneal administration found was 35% and for subcutaneous administration 44% of the bioavailability after intravenous administration.

From the experiments described above it can be concluded that SkQ1 rapidly disappears from the blood. In the next experiment SkQ1 presence in the blood was prolonged by using slow-release infusion pumps introduced subcutaneously into Wistar rats. Blood samples from these animals were taken at timepoints indicated on FIG. 2, and SkQ1 concentration was measured as described above. Results presented in FIG. 2 demonstrate that SkQ1 concentration in the blood can be stabilized using slow release of the compound. This experiment also models administration of SkQ1 using intravenous infusion.

### Example 2. SkQ1 Suppresses Disruption of Blood Vessels Endothelium Monolayer Caused by SIRS Patient Blood Serum Samples

Mitochondrially targeted antioxidant SkQ1 prevented deconstruction of cell-to-cell contacts between human vascular endothelial cells in culture when these cells were treated with blood serum samples from patients with systemic inflammatory response syndrome (SIRS) or sepsis. Disruption of these contacts leads to endothelial damage typically associated with the development of critical condition of a patient. SkQ1 was found to suppress the response to inflammation of vascular endothelium.

Blood samples were taken from patients ages 18-75 years with diagnosed SIRS. Cell cultures of human endothelial cells were pretreated with different amounts of SkQ1, after which the cultures were treated with 5-10% serum from the SIRS blood samples. A one hour VE-cadherin study was performed by fluorescent microscopy followed by western-blot analysis. Results of the experiment are presented on FIG. 3 (western blot) and FIG. 4 (microscopy). These results demonstrate the action of SkQ1 in preventing damage to vascular endothelium during systemic inflammation.

### Example 3. SkQ1 Increases Survival and Symptom-Free Period in Mouse Model of Severe Viral Infection

Mitochondrially targeted antioxidant SkQ1 delayed both development of bad health condition and death caused by influenza virus H5N1 infection in mice.

At day zero, balb\c mice (10-12 g) were inoculated via nasal injection by H5N1 influenza virus (strain chicken/Kurgan/5/2005). Animals were divided into 4 experimental groups:

1. Placebo (n=11)

2. SkQ1 333 nmoles\kg (n=9)

3. SkQ1 1000 nmoles\kg (n=9)

4. SkQ1 3000 nmoles\kg (n=11).

Starting from day 2 (third day after infection) each animal was treated by i/p injection of 450 μl of saline containing the corresponding dosage of SkQ1 or placebo. Time of critical condition development and time of animal death were monitored.

Results of the experiment are presented in FIG. 5 (critical condition) and FIG. 6 (death). These results indicate that SkQ1 treatment can protect an organism from development of critical condition and death caused by severe viral infection.

### Example 4. SkQ1 Toxicology Study: Acute and Chronic Subcutaneous Administration

The acute toxicity of SkQ1 solution administered by subcutaneous administration was studied in SPF male and female CD-1 mice. Lethal effect of high doses of SkQ1 was observed during the first hours after administration due to respiratory arrest, while signs of hepato- and nephrotoxicity were found in mice surviving 14 days after administration of SkQ1 toxic doses. The LD50 was estimated as 31 (20.5-37.2) mg SkQ1/kg in males and 26.8 (23-31) mg SkQ1/kg in females.

SkQ1 solution 28-day chronic toxicity with 14-day recovery was studied in SPF male and female CD-1 mice at SkQ1 dosage of 0.6, 2.5, and 10 mg/kg administered subcutaneously, once daily. At 2.5 mg SkQ1/kg no adverse effects were identified (NOAEL) either at the end of the dosing or at the end of recovery. At 10 mg/kg≈15% animals died due to peritonitis and displayed alterations of diverse systems, most of which were reversed over the 14 days of recovery. Peritonitis stemmed from the local ulcerative action of the formulation, while the adverse reactions of systems/organs were apparently the consequences of peritonitis.

### Example 5. SkQ1 Pretreatment Protects Mice from Death Caused by Intravenous Injection of Mitochondria, Cold Shock, or Toxic Levels of C12 TPP

SkQ1 protected mice in three different mouse models of severe critical conditions. The first critical condition was induced by injection of a suspension of isolated liver mitochondria into the mouse tail vein. The second critical condition was induced by the sharp cold shock of placing mice for 90 minutes in a −20° C. environment. The third condition was induced by administration of a toxic concentration the of lipophilic cation C12 TPP, whose formula is shown below

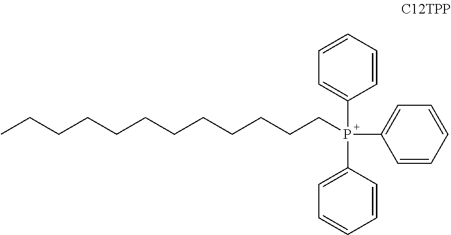

The injection of mitochondria into the bloodstream is perceived by the mammalian body as a signal of severe infection, and the body's response resembles septic shock, as if the blood stream was infected with bacteria or viruses. However, no infectious agents are present in the blood after intravenous administration of mitochondria suspension. Phenomena of this kind are called Damage-Associated Molecular Patterns (DAMPs), referring to release of pro-inflammatory endogenous danger molecules released from damaged cells in response to mechanical damage. This is in contrast to Pathogen-Associated Molecular Patterns (PAMPs) in which pro-inflammatory molecules are released in response to microbial infection. Mitochondrially derived DAMPs have evolutionarily conserved similarities to bacterial PAMPs, and can therefore induce an inflammatory state similar to septic shock. The presence of mitochondria in the blood stream can occur due to activation of the immune system (for example due to viral infection) which forms pores in cell membrane, thus leading to cell lysis or extraction of cell organelles including mitochondria into the blood stream.

FIG. 7 shows the results of an experiment with the injection of isolated mouse liver mitochondria into the tail vein of mice. The injection of mitochondria had no effect on survival during the first day (100% of the animals survived). However only 60% of animals survived the next two days. This survival rate persisted for at least 16 days. A cohort of mice that received the antioxidant SkQ1 for five days before mitochondria injection and then five days after the injection, demonstrated much higher survival (90% of animals survived). See FIG. 7. In this experiment a strong reduction in weight was clearly observed in the first 3 days after injection of mitochondria. Then the surviving animals began to recover—mortality disappeared, and body weight began to increase. In a control experiment, C12 TPP was used instead of SkQ1. In the case of C12 TPP, 20% of mice died in the first half day after the injection of mitochondria, and in two days the mortality had grown to 80%.

In the next experiment, critical condition was caused by a completely different stress, namely by cold shock resulting from incubating mice for 1.5 hours at −20° C. It can be seen in FIG. 8 that cold shock led to dramatic increase of mortality between days 5 and 8, resulting in a survival rate of 40%. SkQ1 premedication and after-shock treatment completely prevented death of the animals, while C12 TPP premedication and treatment increased the mortality to 100% by the sixth day.

In the next experiment a critical condition in mice was induced by i/v injection of a toxic dose of C12 TPP (34 μmole/kg). Considering the membrane-penetrating and membrane-disruptive properties of C12 TPP, it can be expected that the lethal effect of i/v injection of C12 TPP occurs due to disruption of cells in the vascular system, including vascular endothelial cells. FIG. 9 shows that injection of 34 μmol/kg of C12 TPP resulted in 90% mortality within 1 day, and this mortality was prevented by pretreatment of the mice with SkQ1; this occurs by the same mechanism as the prevention of mortality caused by cold shock or i/v injection of mitochondria.

### Example 6. SkQ1 Posttreatment Protects Mice from Death Caused by Intravenous Injection of Mitochondria, Cold Shock, or Toxic Levels of C12 TPP

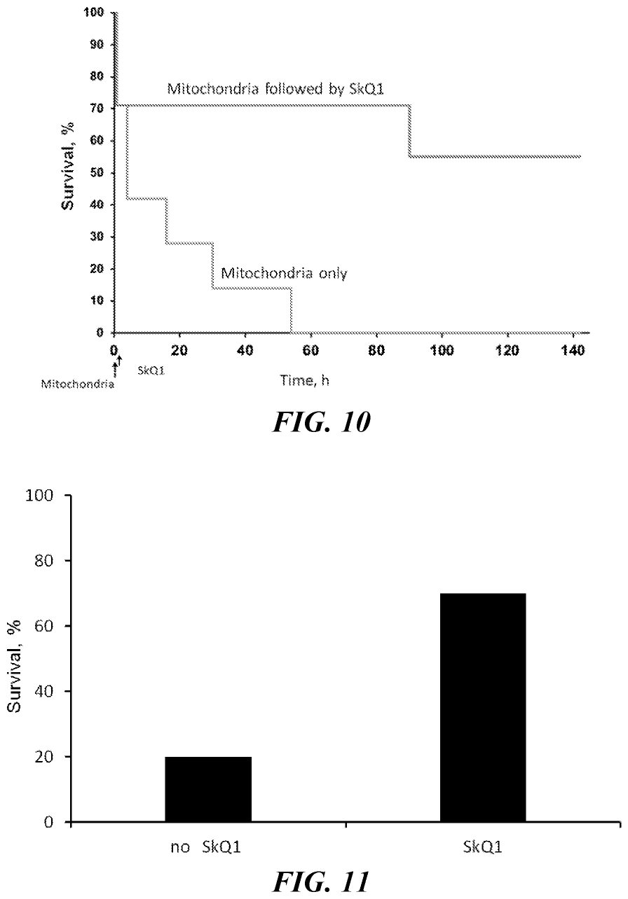

In the previous experiments, it was demonstrated that the SkQ1 premedication protects mice from shock caused by the appearance of mitochondria in the blood, sudden cooling of the body, or injection of C12 TPP. In the next series of experiments, it was investigated whether animals can be rescued by SkQ1 treatment administered simultaneously with the challenge or even after the challenge. As can be seen in FIGS. 10 and 11, mortality from the injection of mitochondria into the bloodstream (FIG. 10) or from cold shock (FIG. 11) was significantly reduced by injection of SkQ1 essentially immediately (one minute) after induction of the shock stimulus.

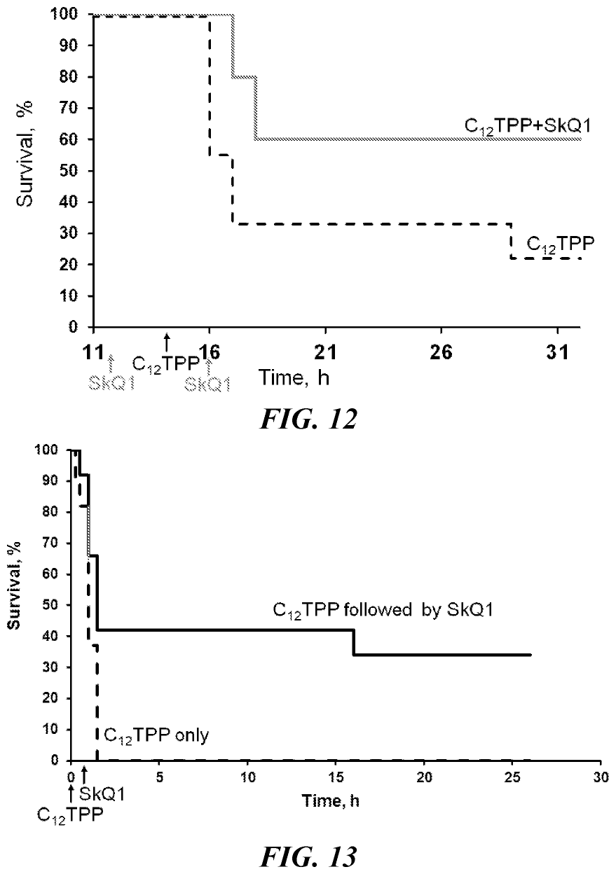

This set of experiments was continued using the lethal shock was caused by toxic concentrations of C12 TPP. Two dosages of C12 TPP were used: 34 μmol/kg C12 TPP (FIG. 12) or 42 μmol/kg C12 TPP (FIG. 13). In the data shown in FIG. 12, SkQ1 (1.5 μmol/kg dosage) was administered to each of the mice both at 4.5 hours before C12 TPP and 0.5 hours after C12 TPP administration. Without SkQ1, about 80% of mice died within 32 hours after the injection of C12 TPP; with SkQ1 the death rate was reduced to 40%. In the experiment described in FIG. 13, the amount of C12 TPP was increased to 42 μmol/kg. As can be seen in FIG. 13, all 12 animals died within 1.5 hours after such treatment in the control group. In the experimental group, SkQ1 was administered once within 1 hour after C12 TPP treatment. In the SkQ1 treatment group ⅓ of the animals survived (FIG. 13).

### Example 7. Effect of SkQ1 on Cytokines TNFα and IL-6

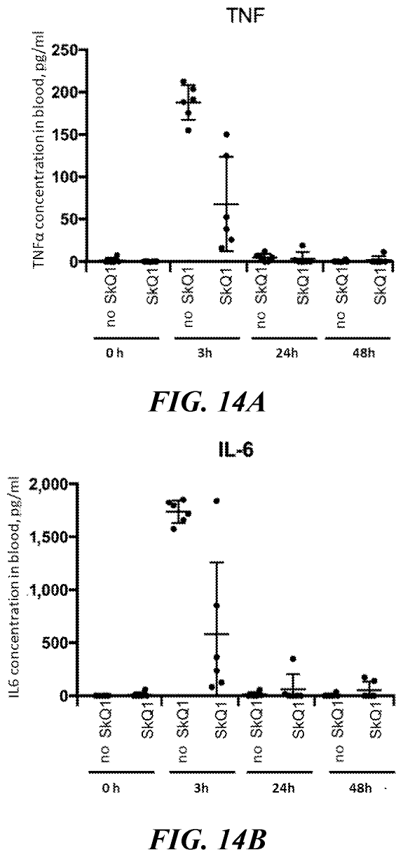

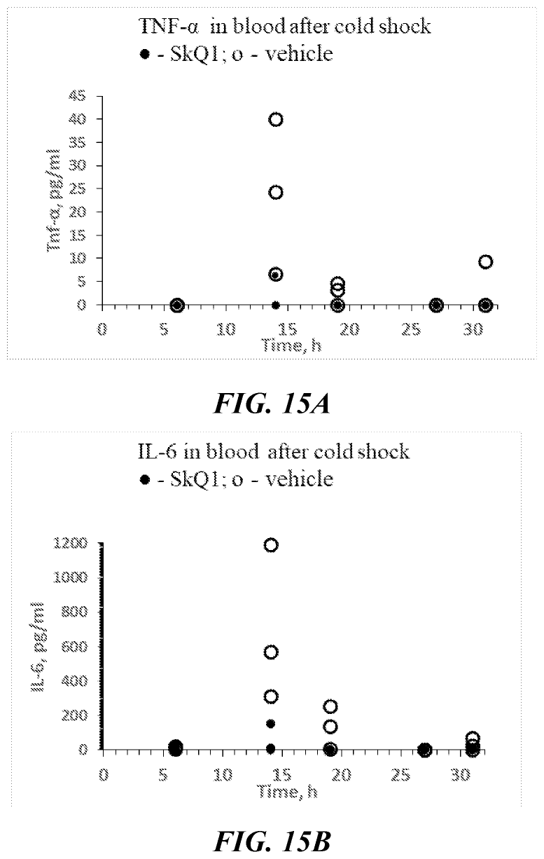

It has been shown that inflammation during coronavirus infection is stimulated by a number of cytokines. In particular, TNFα and IL −6 are considered markers for the presence of inflammation. In this experiment intravenous injection of liver mitochondria caused a significant increase of the blood concentrations of TNFα and of IL −6 in mice. This effect, which reached a maximum about 3 hours after the injection of mitochondria, was almost completely suppressed by intraperitoneal injection of SkQ1 (FIG. 14A and FIG. 14B). Similar results were obtained in the experiment with cold shock (FIG. 15A and FIG. 15B). These results indicate that SkQ not only protects the organism in critical conditions associated with cytokine storm, but also is able to suppress key elements of the cytokine storm.

1. A method to aid in treating or preventing an inflammatory condition in a subject in need thereof, the method comprising administration of a mitochondrially-targeted antioxidant of formula I (SkQ) 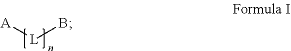
wherein A is an effector moiety/antioxidant optionally having the following structure: 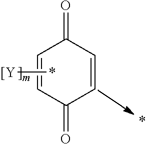
and/or a reduced form thereof, wherein m is an integer from 1 to 3; each Y is independently selected from the group consisting of C₁₋₆ alkyl, C₁₋₆ alkoxy, or two adjacent Y groups, together with carbon atoms to which they are attached, forming the following structure: 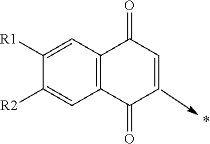
and/or a reduced form thereof, wherein R1 and R2 may be the same or different and are each independently C₁₋₆ alkyl or C₁₋₆ alkoxy;
wherein L is a linker group, comprising:
a) straight or branched hydrocarbon chain which can be optionally substituted by one or more substituents and optionally contains one or more double or triple bonds; and/or
b) a natural isoprene chain;
wherein n is integer from 1 to 40;
wherein B is a mitochondria-targeting group comprising a lipophilic cation and a pharmacologically acceptable anion;
and/or solvates, salts, isomers, or prodrugs thereof.

2. The method of claim 1 wherein SkQ is compound SkQ1 shown below, in its reduced or oxidized form: 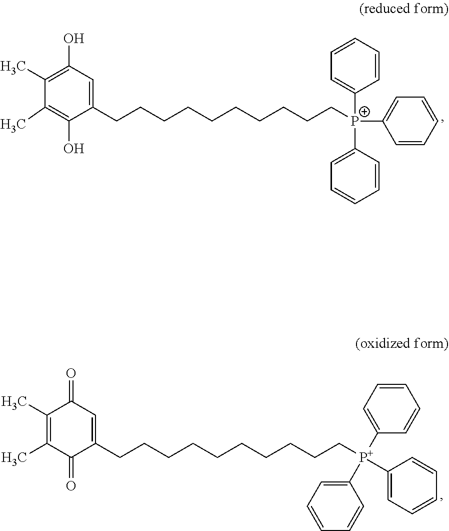 [SMILES: Cc1c(O)cc(CCCCCCCCCC[P+](c2ccccc2)(c2ccccc2)c2ccccc2)c(O)c1C ; CC1=C(C)C(=O)C(CCCCCCCCCC[P+](c2ccccc2)(c2ccccc2)c2ccccc2)=CC1=O]
or a combination thereof.

3. The method of claim 1, wherein SkQ is a compound selected from the group consisting of the following compounds:

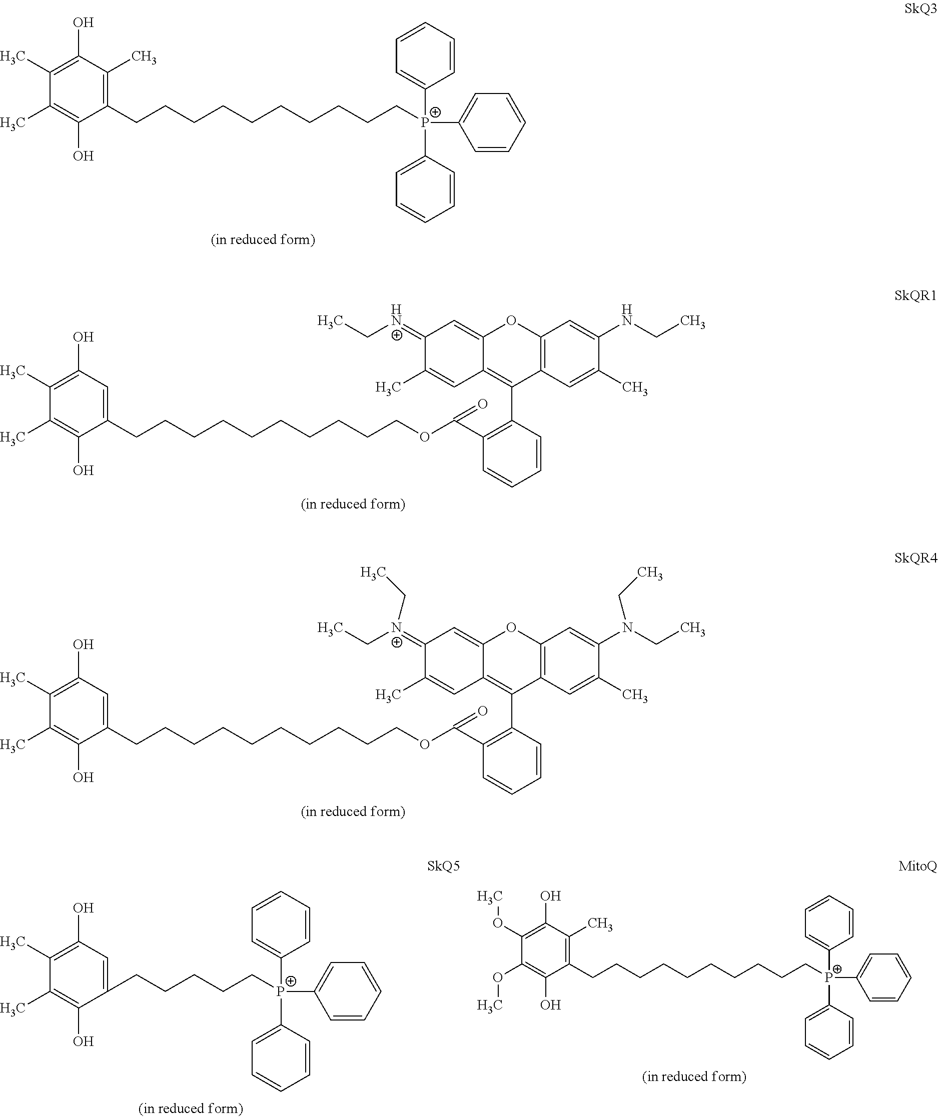

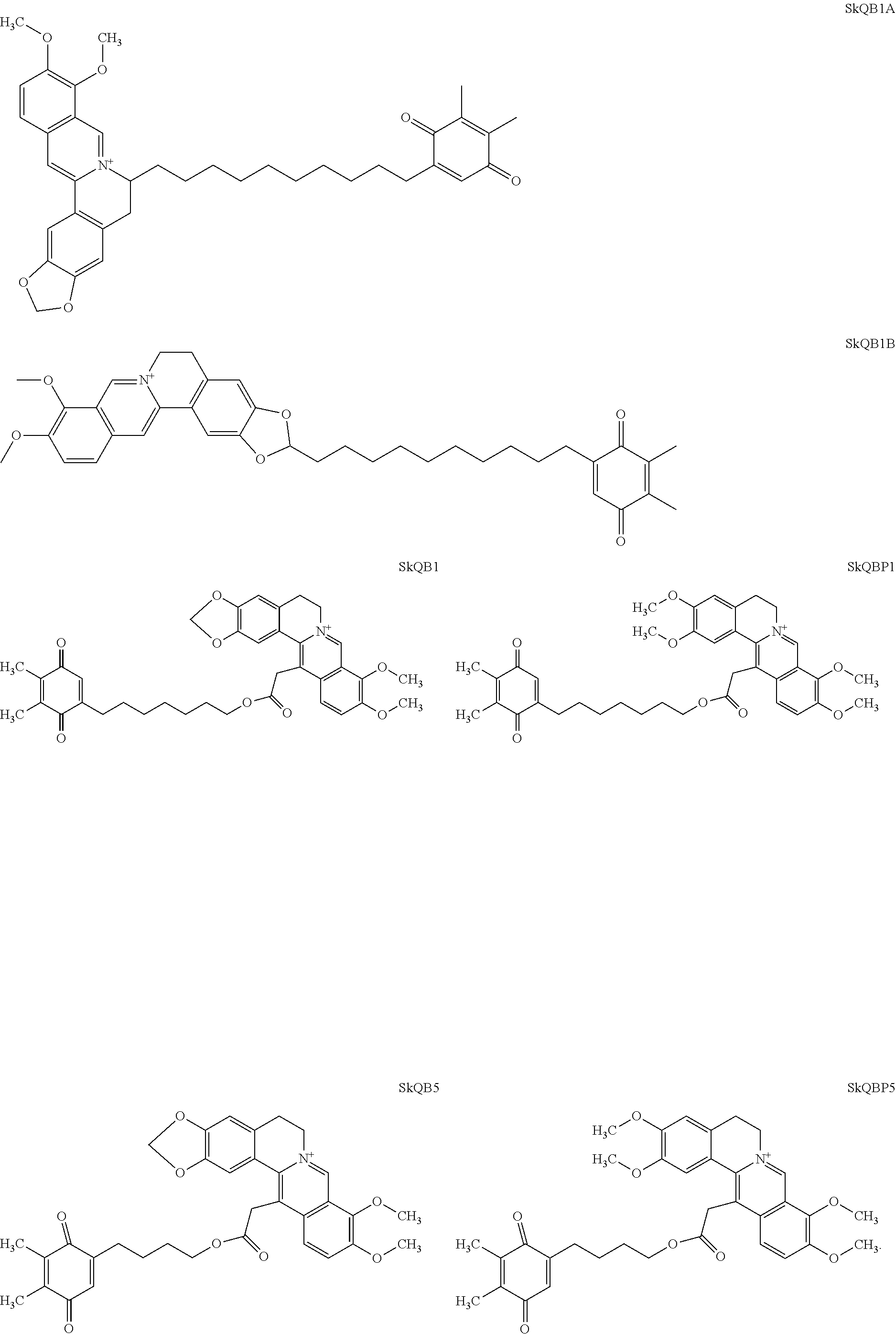

4. The method of any of the preceding claims, wherein the inflammatory condition is associated with infection, COVID-19, sepsis or septic shock, cytokine storm or SIRS, vascular endothelial damage, activation of vascular endothelial cells, PAMPS, DAMPS, cold shock, heat shock, toxic shock, burn trauma, surgery, autoimmune disease, anaphylaxis, cancer, ischemic disease, or presence of CIRP and/or HMGB1 in the blood of the subject.

5. The method of claim 4, wherein the infection is bacterial infection, fungal infection, or viral infection.

6. The method of claim 5, wherein the viral infection is infection by an influenza virus or a corona virus, such as SARS-CoV-2.

7. The method of any of the preceding claims, wherein SkQ is administered by intravenous injection or infusion, subcutaneous injection, or a slow release formulation or device.

8. The method of any of the preceding claims, wherein the method prevents deconstruction of cell-to-cell contacts between endothelial cells of the subject.

9. The method of any of the preceding claims, further comprising administering an additional therapeutic agent.

10. The method of any of the preceding claims, wherein the method prevents the subject from contracting COVID-19.

11. A kit comprising mitochondrially-targeted antioxidant SkQ of formula I below and instructions for performing the method of any of the preceding claims: 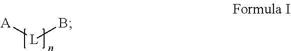
wherein A is an effector moiety/antioxidant optionally having the following structure: 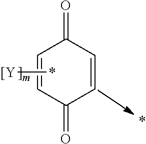
and/or a reduced form thereof, wherein m is an integer from 1 to 3; each Y is independently selected from the group consisting of C₁₋₆ alkyl, C₁₋₆ alkoxy, or two adjacent Y groups, together with carbon atoms to which they are attached, forming the following structure: 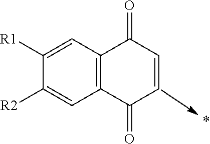
and/or a reduced form thereof, wherein R1 and R2 may be the same or different and are each independently C₁₋₆ alkyl or C₁₋₆ alkoxy;
wherein L is a linker group, comprising:
a) straight or branched hydrocarbon chain which can be optionally substituted by one or more substituents and optionally contains one or more double or triple bonds; and/or
b) a natural isoprene chain;
wherein n is integer from 1 to 40;
wherein B is a mitochondria-targeting group comprising a lipophilic cation and a pharmacologically acceptable anion;
and/or solvates, salts, isomers, or prodrugs thereof.

12. A composition for use in treating or preventing an inflammatory condition in a subject in need thereof, the composition comprising a mitochondrially-targeted antioxidant of formula I (SkQ) 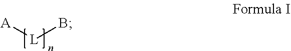
wherein A is an effector moiety/antioxidant optionally having the following structure: 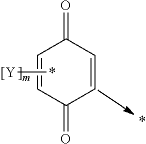
and/or a reduced form thereof, wherein m is an integer from 1 to 3; each Y is independently selected from the group consisting of C₁₋₆ alkyl, C₁₋₆ alkoxy, or two adjacent Y groups, together with carbon atoms to which they are attached, forming the following structure: 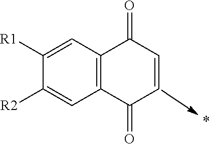
and/or a reduced form thereof, wherein R1 and R2 may be the same or different and are each independently C₁₋₆ alkyl or C₁₋₆ alkoxy;
wherein L is a linker group, comprising:
a) straight or branched hydrocarbon chain which can be optionally substituted by one or more substituents and optionally contains one or more double or triple bonds; and/or
b) a natural isoprene chain;
wherein n is integer from 1 to 40;
wherein B is a mitochondria-targeting group comprising a lipophilic cation and a pharmacologically acceptable anion;
and/or solvates, salts, isomers, or prodrugs thereof.

13. The composition of claim 12 wherein SkQ is compound SkQ1 shown below, in its reduced or oxidized form: 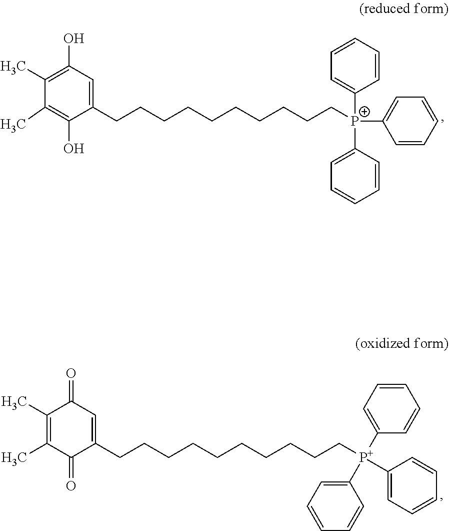 [SMILES: Cc1c(O)cc(CCCCCCCCCC[P+](c2ccccc2)(c2ccccc2)c2ccccc2)c(O)c1C ; CC1=C(C)C(=O)C(CCCCCCCCCC[P+](c2ccccc2)(c2ccccc2)c2ccccc2)=CC1=O]
or a combination thereof.

14. The composition of claim 12, wherein SkQ is a compound selected from the group consisting of the following compounds:

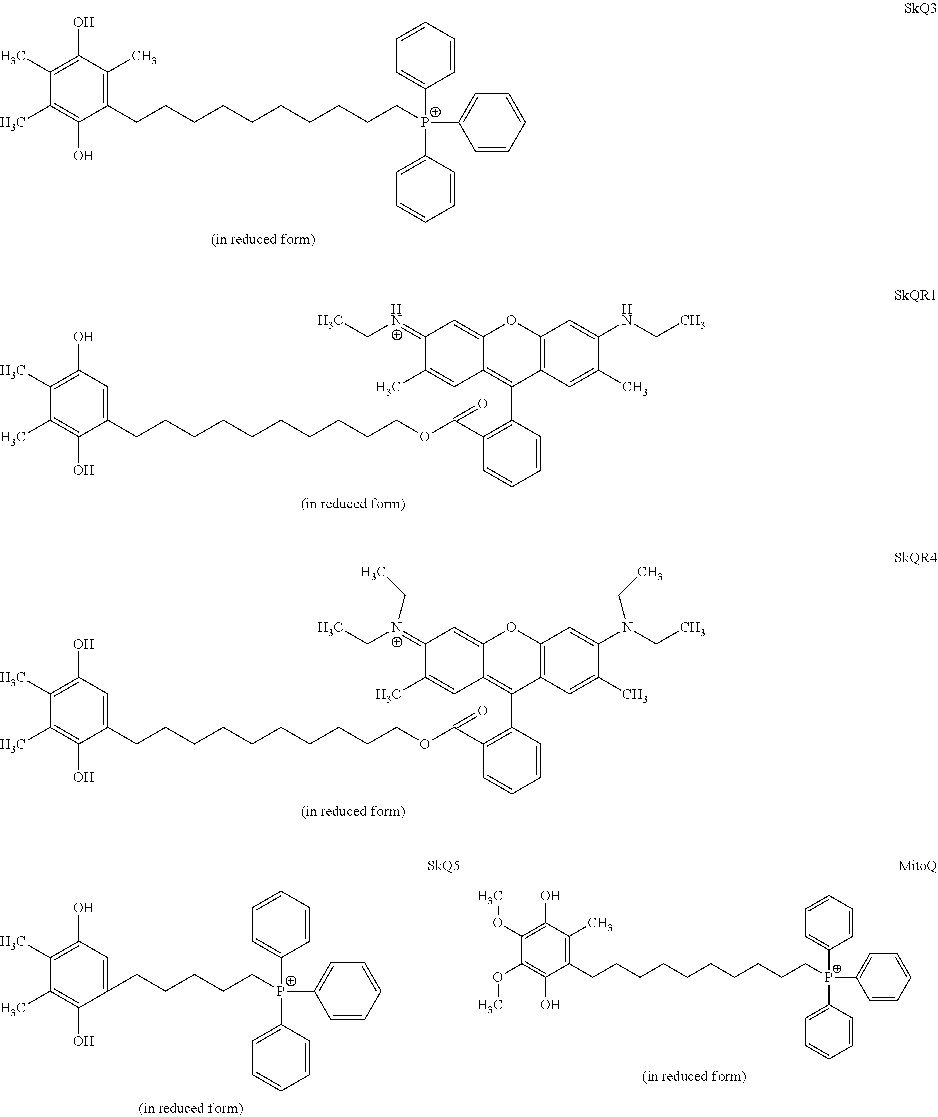

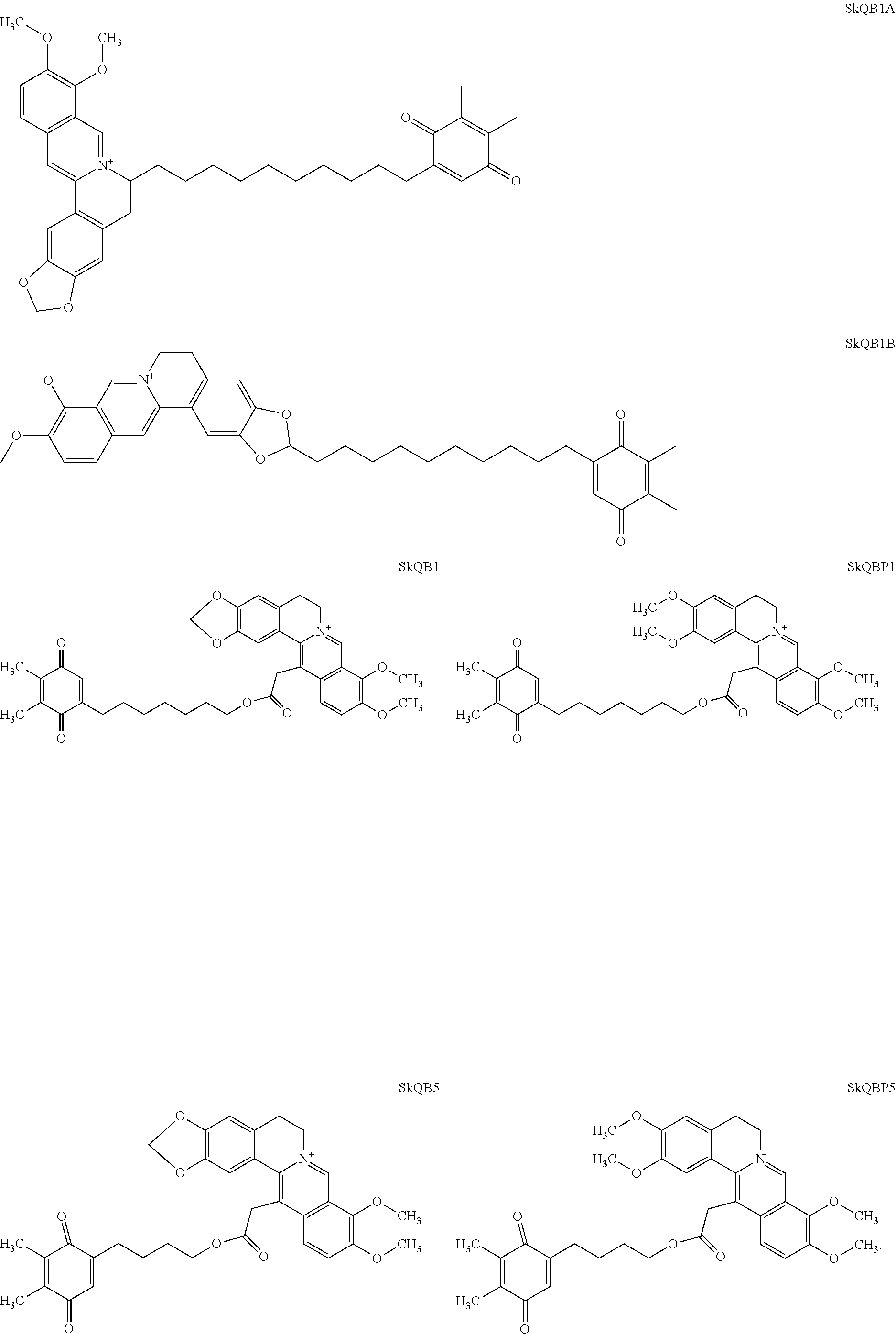

15. The composition of any of claims 12-14, wherein the inflammatory condition is associated with infection, COVID-19, sepsis or septic shock, cytokine storm or SIRS, vascular endothelial damage, activation of vascular endothelial cells, PAMPS, DAMPS, cold shock, heat shock, toxic shock, burn trauma, surgery, autoimmune disease, anaphylaxis, cancer, ischemic disease, or presence of CIRP and/or HMGB1 in the blood of the subject.

16. The method of claim 15, wherein the infection is bacterial infection, fungal infection, or viral infection.

17. The method of claim 16, wherein the viral infection is infection by an influenza virus or a corona virus, such as SARS-CoV-2.
